(19) United States
(12) Patent Application Publication
(10) Pub. No.: US 2025/0144245 A1
(43) Pub. Date: May 8, 2025

(54) GENE THERAPY FOR ARRHYTHMOGENIC CARDIOMYOPATHY

(71) Applicant: Ginkgo Bioworks, Inc., Boston, MA (US)

(72) Inventors: John Graef, Boston, MA (US); Chicheng Sun, Boston, MA (US)

(21) Appl. No.: 18/838,928

(22) PCT Filed: Feb. 17, 2023
(86) PCT No.: PCT/US2023/062834

§ 371 Date: Aug. 15, 2024

Related U.S. Application Data
(60) Provisional application No. 63/383,639, filed on Nov. 14, 2022.
(60) Provisional application No. 63/311,840, filed on Feb. 18, 2022.

(51) Int. Cl. A61K 48/00; C07K 14/47; C12N 15/86

(52) U.S. Cl. CPC A61K 48/0058; A61K 48/0066; C07K 14/4716; C12N 15/86; C12N 2750/14143; C12N 2830/42; C12N 2830/50

(57) ABSTRACT

The disclosure provides nucleic acids (comprising AAV expression cassettes), AAV vectors, and compositions for use in methods for treating and/or delaying the onset of diseases associated with mutations in genes, such as PKP2, associated with arrhythmogenic cardiomyopathy. Also, provided herein are methods for treating and/or delaying the onset of arrhythmogenic cardiomyopathy.

### CROSS-REFERENCE TO RELATED APPLICATIONS

This application claims the benefit of priority to U.S. Provisional Application No. 63/383,639, filed on Nov. 14, 2022, and U.S. Provisional Application No. 63/311,840, filed on Feb. 18, 2022, the contents of which are hereby incorporated by reference in their entireties for all purposes.

### INCORPORATION OF THE SEQUENCE LISTING

The contents of the text file submitted electronically herewith are incorporated herein by reference in their entirety: A computer readable format copy of the Sequence Listing (STRD_026_02WO_SeqList_ST26.xml, date recorded: Feb. 6, 2023, file size: about 44,684 bytes).

### BACKGROUND

Arrhythmogenic cardiomyopathy is a rare familial disorder that usually appears in adulthood, and may cause ventricular tachycardia (fast heart rate) and sudden cardiac death in young, apparently healthy individuals. The clinical hallmark of the disease is ventricular arrhythmias (abnormal heartbeat), arising predominantly from the right ventricle. The pathological hallmark of the disease is fibrofatty replacement of right ventricular myocardium. Symptoms commonly include a sensation of fluttering or pounding in the chest (palpitations), light-headedness, and fainting (syncope). Over time, patients may experience shortness of breath and abnormal swelling in the legs or abdomen. If the myocardium becomes severely damaged in the later stages of the disease, it can lead to heart failure.

Arrhythmogenic cardiomyopathy is often caused by mutations in genes that encode proteins, such as, plakophilin-2, which are part of desmosomes. Desmosomes are specialized adhesive protein complexes that localize to intercellular junctions, which promote attachment between heart muscle cells, thereby maintaining the integrity of myocardial tissue. Mutations in genes encoding desmosomal proteins impair the function of desmosomes, resulting in damage to the myocardium and replacement of cardiac tissue with fat or fibrotic tissue.

Standard of care for arrhythmogenic cardiomyopathy comprises management of symptoms using medication (such as, beta blockers and amiodarone), implantable cardioverter defibrillators (ICDs) and catheter ablation. Thus, there is a persistent need for disease-modifying therapeutic compositions and methods to treat arrhythmogenic cardiomyopathy.

### SUMMARY

The disclosure provides nucleic acid molecules, comprising an adeno-associated virus (AAV) expression cassette, wherein the AAV expression cassette comprises, from 5′ to 3′: (i) a 5′ AAV inverted terminal repeat (ITR); (ii) a promoter; (iii) an arrhythmogenic cardiomyopathy-associated transgene; and (iv) a 3′ AAV ITR. In some embodiments, the promoter is capable of expressing the transgene in a cardiac cell. In some embodiments, the promoter comprises a cardiac troponin T (TNNT2) promoter, such as, for example, a TNNT2 promoter comprising the nucleic acid sequence SEQ ID NO: 4, or a sequence at least 90% identical thereto. In some embodiments, the transgene encodes a plakophilin-2, such as, for example, a transgene comprising the nucleic acid sequence of SEQ ID NO: 2, or a sequence at least 90% identical thereto. In some embodiments, the AAV expression cassette comprises a nucleic acid sequence SEQ ID NO: 12, or a sequence at least 90% identical thereto.

The disclosure further provides plasmids, comprising any one of the nucleic acid molecules disclosed herein; and cells, comprising any one of the nucleic acid molecules or plasmids disclosed herein. Furthermore, the disclosure provides methods of producing recombinant AAV vectors, comprising contacting AAV producer cells with any one of the nucleic acid molecules or plasmids disclosed herein. The disclosure also provides recombinant AAV vectors produced by any one of the methods of producing recombinant AAV vectors disclosed herein. In some embodiments, the recombinant AAV vector is a single-stranded AAV (ssAAV). In some embodiments, the recombinant AAV vector is a self-complementary AAV (scAAV). In some embodiments, the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

In some embodiments, the AAV vector comprises a capsid protein with one or more substitutions or mutations, as compared to a wild type AAV capsid protein. For instance, in some embodiments, the AAV vector comprises a capsid protein comprising: (i) the amino acid sequence of SEQ ID NO: 13, or a sequence at least 90% identical thereto, or (ii) the amino acid sequence of SEQ ID NO: 14, or a sequence at least 90% identical thereto, or (iii) the amino acid sequence of SEQ ID NO: 15, or a sequence at least 90% identical thereto. In some embodiments, the AAV vector comprises a capsid protein comprising the amino acid sequence of SEQ ID NO: 13, or a sequence at least 90% identical thereto. In some embodiments, the AAV vector comprises a capsid protein comprising the amino acid sequence of SEQ ID NO: 13. In some embodiments, the AAV vector comprises a capsid protein comprising the amino acid sequence of SEQ ID NO: 14, or a sequence at least 90% identical thereto. In some embodiments, the AAV vector comprises a capsid protein comprising the amino acid sequence of SEQ ID NO: 14. In some embodiments, the AAV vector comprises a capsid protein comprising the amino acid sequence of SEQ ID NO: 15, or a sequence at least 90% identical thereto. In some embodiments, the AAV vector comprises a capsid protein comprising the amino acid sequence of SEQ ID NO: 15. The disclosure further provides compositions, comprising any one of the nucleic acids, any one of the plasmids, any one of the cells, or any one of the recombinant AAV vectors disclosed herein, and a pharmaceutically acceptable carrier.

The disclosure also provides methods of expressing an arrhythmogenic cardiomyopathy-associated transgene in a cell, comprising: contacting the cell with any one of the nucleic acids, plasmids, cells, recombinant AAV vectors, or compositions disclosed herein, thereby expressing the arrhythmogenic cardiomyopathy-associated transgene in the cell.

The disclosure also provides methods of expressing an arrhythmogenic cardiomyopathy-associated transgene in a tissue, comprising: contacting the tissue with any one of the nucleic acids, plasmids, cells, recombinant AAV vectors, or compositions disclosed herein, thereby expressing the arrhythmogenic cardiomyopathy-associated transgene in the tissue. In some embodiments, the tissue comprises at least one cell, and at least one desmosomal junction.

In some embodiments, the cell is a cardiac cell, an endothelial cell, a skin cell, a bladder cell, or a gastrointestinal mucosal cell. In some embodiments, the cell is a cardiac cell. In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the recombinant AAV vectors, or the composition to the subject. In some embodiments, the subject suffers from, or is at a risk of developing the arrhythmogenic cardiomyopathy.

The disclosure also provides methods of treating arrhythmogenic cardiomyopathy in a subject in need thereof, comprising administering to the subject a therapeutically effective amount of any one the nucleic acids, plasmids, cells, recombinant AAV vectors, or compositions disclosed herein, thereby treating arrhythmogenic cardiomyopathy in the subject. In some embodiments, the arrhythmogenic cardiomyopathy is arrhythmogenic right ventricular cardiomyopathy. In some embodiments, the arrhythmogenic cardiomyopathy is associated with, promoted by, or caused by a genetic mutation. In some embodiments, the genetic mutation comprises a mutation in the PKP2 gene. In some embodiments, the mutation in the PKP2 gene results in PKP2 haploinsufficiency.

In some embodiments, the method comprises diminishing the severity of; delaying the onset or progression of; and/or eliminating a symptom of the arrhythmogenic cardiomyopathy. In some embodiments, the symptom of the arrhythmogenic cardiomyopathy comprises: (a) re-entrant ventricular tachycardia, (b) syncope, (c) sudden death, or (d) any combination thereof. In some embodiments, the arrhythmogenic cardiomyopathy is associated with: (a) decreased mechanical stability between cardiomyocytes of the subject, (b) disruption of gap junctions in the cardiac tissue of the subject, (c) decreased sodium currents in the cardiac tissue of the subject, (d) fibrosis of the right ventricular myocardium, or (e) any combination thereof.

In some embodiments, the method comprises increasing the mechanical stability between cardiomyocytes of the subject. In some embodiments, the method comprises improving the function of gap junctions in the cardiac tissue of the subject. In some embodiments, the method comprises increasing sodium currents in the cardiac tissue of the subject. In some embodiments, the method comprises decreasing fibrosis of the right ventricular myocardium of the subject.

In some embodiments, the subject is a human subject. In some embodiments, the nucleic acid molecule, the plasmid, the cell, the recombinant AAV vector, or composition is administered to the cardiac tissue of the subject. In some embodiments, the nucleic acid molecule, the plasmid, the cell, the recombinant AAV vector, or composition is administered to the left atrium, right atrium, left ventricle, right ventricle and/or septum. In some embodiments, the nucleic acid molecule, the plasmid, the cell, the recombinant AAV vector, or composition is administered to the subject, via intravenous administration, intra-arterial administration, intra-aortic administration, direct cardiac injection, coronary artery perfusion, or any combination thereof.

These and other embodiments are addressed in more detail in the detailed description set forth below.

### BRIEF DESCRIPTION OF FIGURES

The patent or application file contains at least one drawing executed in color. Copies of this patent or patent application publication with color drawing(s) will be provided by the Office upon request and payment of the necessary fee.

FIG. 1 shows a schematic of an AAV expression cassette of SEQ ID NO: 12, comprising the following elements: (i) a 5′ AAV ITR of SEQ ID NO: 5, (ii) a cardiac troponin T (TNNT2) promoter of SEQ ID NO: 4; (iii) a human beta-globin (hBG) intron of SEQ ID NO: 7; (iv) a human PKP2a transgene sequence of SEQ ID NO: 2; (v) a bovine growth hormone (bGH) polyA signal of SEQ ID NO: 8; and (vi) a 3′ AAV ITR of SEQ ID NO: 6.

FIG. 2 shows the map of a plasmid comprising the AAV expression cassette shown in FIG. 1.

FIG. 3A shows the fold change in PKP2 mRNA expression as compared to a housekeeping gene, UBC, in normal human iPSC-derived cardiomyocytes that were either untreated (“wild type”), treated with an antisense oligonucleotide (ASO) against PKP2 to knock down expression (“PKP2 KD”) or knocked down then treated with 50K MOI of AAV.STRV47-hTNNT2.PKP2a. FIG. 3B shows Western Blot results of PKP2 protein in wild type cells, untreated PKP2 KD cells, and PKP2 KD cells treated with 50K MOI of AAV.STRV47-hTNNT2.PKP2a. FIG. 3C depicts the quantification of the protein band intensity in FIG. 3B.

FIG. 4A shows the results from a Western Bot analysis of PKP2a protein expression in wild type cardiomyocytes, and CRISPR-induced PKP2 knockout (“PKP2-KO”) iPSC cardiomyocytes that were either untreated or transduced with rAAV encoding hTNNT2.PKP2a (AAV.STRV5-hTNNT2.PKP2a). FIG. 4B is a graph showing the quantitation of PKP2a protein expression, normalized to GAPDH in wild type cardiomyocytes, and PKP2-KO iPSC cardiomyocytes that were either untreated or transduced with rAAV encoding hTNNT2.PKP2a (AAV.STRV5-hTNNT2.PKP2a). FIG. 4C shows images from immunofluorescence microscopy of wild type and PKP2-KO iPSC cardiomyocytes, which are either untreated, or transduced with rAAV encoding hTNNT2.PKP2a (AAV.STRV5-hTNNT2.PKP2a).

FIG. 5A is a graph showing the spontaneous calcium transients measured using FLIPR Calcium 6 (SpectraMax® i3x) in wild type iPSC cardiomyocytes transduced with a control AAV.STRV5-Cbh.GFP. FIGS. 5B and 5C are graphs showing the spontaneous calcium transients measured using FLIPR Calcium 6 (SpectraMax® i3x) in PKP2-KO iPSC cardiomyocytes (FIG. 5C) and in PKP2-KO iPSC cardiomyocytes transduced with a control AAV.STRV5-Cbh.GFP (FIG. 5B), indicating that the loss of PKP2 function resulted in early after-transients. FIG. 5D is a graph showing the spontaneous calcium transients measured using FLIPR Calcium 6 (SpectraMax® i3x) in PKP2-KO iPSC cardiomyocytes transduced with AAV.STRV5-hTNNT2.PKP2a indicating that the early after-transients were absent after transduction with AAV.STRV5-hTNNT2.PKP2a.

FIG. 6A is a graph showing the vector copy number (VCN) in cardiac tissues of WT mice 21 days post IV injection via tail vein (5e13vg/kg) of AAV.STRV47-hTNNT2.PKP2a. FIG. 6B is a graph showing the levels of PKP2 mRNA in the cardiac tissues of WT mice 21 days post IV injection via tail vein (5e13vg/kg) of AAV.STRV47-hTNNT2.PKP2a. FIG. 6C is a Western Blot showing the human PKP2 protein in extracts of cardiac tissue of WT mice 21 days post IV injection via tail vein (5e13vg/kg) of AAV.STRV47-hTNNT2.PKP2a. FIG. 6D depicts the quantification of the protein band intensity in FIG. 6C.

FIG. 7 shows the design for studying the treatment of a tamoxifen-induced, cardiac-specific PKP2 knockout mouse model with AAV.STRV5-hTNNT2.PKP2a, or AAV.STRV84-hTNNT2.PKP2a.

FIG. 8A shows the combined overall survival of cohort 1 and cohort 2 for the indicated mice groups over time. See also, Table B. FIG. 8B is a graph showing the probability of survival calculated using GraphPad Prism using a Kaplan-Meier curve of untreated PKP2-cKO mice (n=5); mice with normal PKP2 function—that is, Pkp2 wt/wt; Cre+ (“WT, Cre+”; n=5) and Pkp2 fl/fl; Cre− (FL, Cre−; n=4) controls; and PKP2-cKO mice treated with AAV.STRV5-hTTNT2.PKP2a (n=7). Study was terminated 54 days post tamoxifen injection (dpi).

FIG. 9A is a graph showing the vector copy number (VCN) in the liver and heart tissues of PKP2 cardiac knockout mice 70 days post IV injection via tail vein (5e13vg/kg) of AAV.STRV5-hTNNT2.PKP2a, or AAV.STRV84-hTNNT2.PKP2a. FIG. 9B is a graph showing the levels of PKP2 mRNA in the liver and heart tissues of PKP2 cardiac knockout mice 70 days post IV injection via tail vein (5e13vg/kg) of AAV.STRV5-hTNNT2.PKP2a, or AAV.STRV84-hTNNT2.PKP2a.

FIG. 10A is a Western Blot showing human PKP2 protein bands in the heart tissue extracts of individual Group 2 mice (PKP2 wt/wt; Cre+), Group 3 mice (PKP2 fl/fl; Cre−), Group 1 mice (PKP2 fl/fl; Cre+) and treated PKP2 fl/fl; Cre+ mice 70 days post IV injection via tail vein (5e13vg/kg) of AAV.STRV5-hTNNT2.PKP2a (Group 4), or AAV.STRV84-hTNNT2.PKP2a (Group 5). FIG. 10B shows the total protein loading control. See also, Table E in Example 6.

FIGS. 11A-11E are images showing the localization of PKP2 via immunohistochemistry in PKP2 wt/wt; Cre+ mice (FIG. 11A), PKP2 fl/fl; Cre−, tamoxifen-treated mice (FIG. 11B), PKP2 fl/fl, Cre+ and tamoxifen-treated mice (FIG. 11C), PKP2 fl/fl, Cre+ and tamoxifen-treated mice, which were also treated with either 5e13vg/kg (FIG. 11D), or 5e13vg/kg AAV.STRV84-AAV.STRV5-hTNNT2.PKP2a vector hTNNT2.PKP2a vector (FIG. 11E).

FIGS. 12A-12B are line graphs showing results from biweekly echocardiography quantifying left ventricular ejection fraction (LVEF) (FIG. 12A) and systolic right ventricular area (RV area-S) (FIG. 12B) of control mice that have wild type PKP2 function (WT; Cre+ mice and floxed; Cre− mice), PKP2-cKO mice and PKP2-cKO mice treated with AAV.STRV5-hTNNT2.PKP2a. FIGS. 12C-12D are bar graphs showing a comparison of LVEF (FIG. 12C) and RV area-S (FIG. 12D) in control mice that have wild type PKP2 function (WT; Cre+ mice and floxed; Cre− mice), PKP2-cKO mice and PKP2-cKO mice treated with AAV.STRV5-hTNNT2.PKP2a at 42 dpi. *p<0.05, **p<0.01, ***p<0.001, ***p<0.0001.

FIG. 13A shows a Western Blot showing PKP2 protein bands in the heart tissue extracts of WT (lane 2); antisense-oligonucleotide knockdown in iPSC-derived cardiomyocytes (“−Control KD”) (lane 3); PKP2-cKO mice (“−Control cKO”) (lane 4); cardiomyocytes lysates that have been transfected with AAV.STRV84-hTNNT2.PKP2a (“+Control”) (lanes 5-13); and PKP2-cardiac knockout mice treated with AAV.STRV5-hTNNT2.PKP2a (“Treated”) (lanes 14-20), with the PKP2a protein band highlighted with an arrow (expected human size 92-97 kDa, expected mouse PKP2 size 88 kDa). Coomassie in blue indicates total protein loaded in each lane. FIG. 13B is a graph showing the quantification of PKP2 protein bands seen in a Western blot generated from mouse cardiac tissues of PKP2-cardiac knockout mice (“−Control”); WT, Cret mice (“+Control”); FL, Cre− mice (“+Control”) and PKP2-cardiac knockout mice treated with AAV.STRV5-hTNNT2.PKP2a (“hTNNT2.PKP2a treated”), which shows that treatment of PKP2-cardiac knockout mice with AAV.STRV5-hTNNT2.PKP2a restores PKP2 levels in the heart. FIG. 13C is a graph showing the vector copy number/μg DNA in heart and liver tissues of PKP2-cKO mice treated with AAV.STRV5-hTNNT2.PKP2a, as measured by qPCR. FIG. 13D is a graph showing the PKP2a mRNA transcript levels in heart and liver tissues of PKP2-cKO mice treated with AAV.STRV5-hTNNT2.PKP2a, as measured by RT-qPCR demonstrating tissue-specific expression in heart. ****p<0.0001.

FIGS. 14A-14D are representative histology images from the right ventricles of the heart of PKP2-cKO mice treated with AAV.STRV84-hTNNT2.PKP2a (FIG. 14D) or treated with AAV.STRV5-hTTNT2.PKP2a (FIG. 14C), in comparison to −Control (PKP2-cardiac knockout mice; FIG. 14A) and +Control (FL, Cre− mice; FIG. 14B) animals. Masson's trichrome images (top row) show collagen in blue. Corresponding H&E images are shown (bottom row). Images show reduction of RV fibrosis with hTNNT2.PKP2a treatment.

FIG. 15 shows the quantification of the fibrosis shown in FIGS. 14A-14D. The quantification for FIG. 14A is seen in the −control (PKP2-cKO) column. The quantification for FIG. 14B is seen the +Control (FL, Cre−) column. The quantification for FIG. 14C is seen in the AAV.STRV5-hTNNT2.PKP2a column. The quantification for FIG. 14D is seen in the AAV.STRV84-hTNNT2.PKP2a column. +Control (wt, Cre+) is an additional positive control showing the results of quantification of fibrosis staining in the wild type animal.

FIGS. 16A-16B show the correlation between the dosage and vector copy number in heart or liver of mouse PKP2 (mPKP2 (SEQ ID NO: 20)) (FIG. 16A) and human PKP2 (hPKP2 (SEQ ID NO: 2)) (FIG. 16B) transgenes in AAV particles comprising a capsid protein having the amino acid sequence of SEQ ID NO: 15. The vector copy number in the liver is about 1-2 logs higher than in the heart for both transgenes.

FIGS. 17A-17B show the correlation between dosage and mRNA of mouse PKP2 (FIG. 17A) and human PKP2 (FIG. 17B) transgenes expressed in both the heart and liver.

FIG. 18 shows a Western Blot showing mouse PKP2 protein bands in the heart tissue extracts of the mPKP2-treated mice. WT (Group 1); fl/fl Cre− (Group 2); fl/fl Cre+ (Group 3); ultra-low dose (Group 4); low dose (Group 5); medium dose (Group 6); and high dose (Group 7) with the PKP2a protein band highlighted with an arrow (expected human size 92-97 kDa, expected mouse PKP2 size 88 kDa). Coomassie in blue indicates total protein loaded in each lane.

FIG. 19 shows a Western Blot showing human PKP2 protein bands in the heart tissue extracts of the hPKP2-treated mice. WT (Group 1); fl/fl Cre− (Group 2); fl/fl Cre+ (Group 3); low dose (Group 8); and high dose (Group 9) with the PKP2a protein band highlighted with an arrow (expected human size 92-97 kDa, expected mouse PKP2 size 88 kDa). Coomassie in blue indicates total protein loaded in each lane.

FIG. 20 shows a Western Blot showing mouse PKP2 protein bands in the left lateral liver tissue extracts of the mPKP2-treated mice. WT (Group 1); fl/fl Cre− (Group 2); fl/fl Cre+ (Group 3); ultra-low dose (Group 4); low dose (Group 5); medium dose (Group 6); and high dose (Group 7) with the PKP2a protein band highlighted with an arrow (expected human size 92-97 kDa, expected mouse PKP2 size 88 kDa). Coomassie in blue indicates total protein loaded in each lane.

FIG. 21 shows a Western Blot showing human PKP2 protein bands in the left lateral liver tissue extracts of the hPKP2-treated mice. WT (Group 1); fl/fl Cre− (Group 2); fl/fl Cre+ (Group 3); low dose (Group 8); and high dose (Group 9) with the PKP2a protein band highlighted with an arrow (expected human size 92-97 kDa, expected mouse PKP2 size 88 kDa). Coomassie in blue indicates total protein loaded in each lane.

FIGS. 22A-22B show the increase in the probability of survival for ARVC mice (PKP2 fl/fl; Cre+) that were injected with increasing dosages of the STRV84-mouse PKP2 (FIG. 22A) or STRV84-human PKP2 (FIG. 22B), respectively.

FIGS. 23A-23F show the increase in rescue of the ARVC mice relative to the dose of mPKP2 (ultra-low, low, medium, and high) in the (FIG. 23A) ejection fraction; (FIG. 23B) LV volume; (FIG. 23C) stroke volume; (FIG. 23D) percent fractional shortening; (FIG. 23E) cardiac output; and (FIG. 23F) left ventricular outflow tract velocity time integral (LVOT VTI).

FIGS. 24A-24F show the increase in rescue of the ARVC mice relative to the dose of mPKP2 (low and high) in the (FIG. 24A) ejection fraction; (FIG. 24B) LV volume; (FIG. 24C) stroke volume; (FIG. 24D) percent fractional shortening; (FIG. 24E) cardiac output; and (FIG. 24F) LVOT VTI.

### DETAILED DESCRIPTION

Around 40-60% of patients with arrhythmogenic cardiomyopathy have a mutation in a desmosomal protein-encoding gene, with autosomal dominant mutations in PKP2 (encoding plakophilin-2) being the most prominent (around 50-70%). The estimated prevalence for arrhythmogenic cardiomyopathy with PKP2 haploinsufficiency is about 1:6,000 to about 1:25,000. At a cellular level, reduced or eliminated PKP2 function leads to decreased mechanical stability between cardiomyocytes, disruption of gap junctions (such as, gap junctions comprising connexin 43), and decreased sodium currents. These cellular effects lead to the fibrosis of the right ventricular myocardium, which may result in re-entrant ventricular tachycardia, syncope and sudden death.

PKP2 encodes plakophilin-2, which is one of three plakophilins that are expressed in both cardiac progenitors and differentiated cardiomyocytes. Plakophilin-2 is also expressed in other cell types with desmosomal junctions, such as, endothelial cells. Plakophilin-2 is a desmosomal, structural protein, which links the intermediate filament network in cardiac cells to the intercellular cadherin proteins, desmocollin and desmoglein. Plakophilin-2 also plays a role in recruitment and stabilization of other desmosomal proteins, and this contributes to the integrity and function of the desmosome of cardiac cells, and hence to the integrity and function of the myocardium.

The disclosure provides nucleic acids (comprising AAV expression cassettes), AAV vectors, and compositions for use in methods for treating and/or delaying the onset of diseases associated with mutations in arrhythmogenic cardiomyopathy-associated genes, such as, PKP2. Also, provided herein are methods for treating and/or delaying the onset of arrhythmogenic cardiomyopathy.

### Definitions

The following terms are used in the description herein and the appended claims:

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, ±10%, ±5%, ±1%, ±0.5%, or even ±0.1% of the specified amount.

Also as used herein, “and/or” refers to and encompasses any and all possible combinations of one or more of the associated listed items, as well as the lack of combinations when interpreted in the alternative (“or”).

The term “wild type” is a term of the art understood by skilled persons and means the typical form of an organism, strain, gene, protein, or characteristic as it occurs in nature as distinguished from mutant or variant forms. For example, a wild type protein is the typical form of that protein as it occurs in nature.

The term “mutant protein” is a term of the art understood by skilled persons and refers to a protein that is distinguished from the wild type form of the protein on the basis of the presence of amino acid modifications, such as, for example, amino acid substitutions, insertions and/or deletions. The term “mutant gene” is a term of the art understood by skilled persons and refers to a gene that is distinguished from the wild type form of the gene on the basis of the presence of nucleic acid modifications, such as, for example, nucleic acid substitutions, insertions and/or deletions. In some embodiments, the mutant gene encodes a mutant protein.

A “nucleic acid” or “polynucleotide” is a sequence of nucleotide bases, for example RNA, DNA or DNA-RNA hybrid sequences (including both naturally occurring and non-naturally occurring nucleotides). In some embodiments, the nucleic acids of the disclosure are either single or double stranded DNA sequences. A nucleic acid may be 1-1,000, 1,000-10,000, 10,000-100,000, 100,000-1 million or greater than 1 million nucleotides in length. A nucleic acid will generally contain phosphodiester bonds, although in some cases nucleic acid analogs are included that may have alternate backbones, comprising, for example, phosphoramide, phosphorothioate, phosphorodithioate, O-methylphophoroamidite linkages, and peptide nucleic acid backbones and linkages. Other analog nucleic acids include those with positive backbones, non-ionic backbones, and non-ribose backbones. Nucleic acids containing one or more carbocyclic sugars are also included within the definition of nucleic acids. These modifications of the ribose-phosphate backbone may facilitate the addition of labels, or to increase the stability and half-life of such molecules in physiological environments. Nucleic acids of the disclosure may be linear, or may be circular (e.g., a plasmid).

As used herein, the term “promoter” refers to one or more nucleic acid control sequences that direct transcription of an operably linked nucleic acid. Promoters may include nucleic acid sequences near the start site of transcription, such as a TATA element. Promoters may also include cis-acting polynucleotide sequences that can be bound by transcription factors.

A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

An “AAV expression cassette” is a nucleic acid that gets packaged into a recombinant AAV vector, and comprises a sequence encoding one or more transgenes. When the AAV vector is contacted with a target cell, the transgenes are expressed by the target cell.

As used herein, the terms “virus vector,” “viral vector,” or “gene delivery vector” refer to a virus particle that functions as a nucleic acid delivery vehicle, and which comprises a nucleic acid (e.g., an AAV expression cassette) packaged within a virion. Exemplary virus vectors of the disclosure include adenovirus vectors, adeno-associated virus vectors, lentivirus vectors, and retrovirus vectors.

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV218, AAV2g9, AAV-LK03, AAV7m8, AAV Anc80, AAV PHP.B, and any other AAV now known or later discovered. See, e.g., Table 1.

TABLE 1
Adeno-Associated Virus Serotypes

| | GenBank | | GenBank | | GenBank |
|---|---|---|---|---|---|
| | Accession | | Accession | | Accession |
| | Number | | Number | | Number |
| Complete | | Clade C | | Rh57 | AY530569 |
| Genomes | | | | | |
| Adeno- | NC_002077, | Hu9 | AY530629 | Rh50 | AY530563 |
| associated virus 1 | AF063497 | | | | |
| Adeno- | NC_001401 | Hu10 | AY530576 | Rh49 | AY530562 |
| associated virus 2 | | | | | |
| Adeno- | NC_001729 | Hu11 | AY530577 | Hu39 | AY530601 |
| associated virus 3 | | | | | |
| Adeno- | NC_001863 | Hu53 | AY530615 | Rh58 | AY530570 |
| associated virus 3B | | | | | |
| Adeno- | NC_001829 | Hu55 | AY530617 | Rh61 | AY530572 |
| associated virus 4 | | | | | |
| Adeno- | Y18065, | Hu54 | AY530616 | Rh52 | AY530565 |
| associated virus 5 | AF085716 | | | | |
| Adeno- | NC_001862, | Hu7 | AY530628 | Rh53 | AY530566 |
| associated virus 6 | AAB95450.1 | | | | |
| Avian AAV | AY186198, | Hu18 | AY530583 | Rh51 | AY530564 |
| ATCC VR-865 | AY629583, | | | | |
| | NC_004828 | | | | |
| Avian AAV | NC_006263, | Hu15 | AY530580 | Rh64 | AY530574 |
| strain DA-1 | AY629583 | | | | |
| Bovine AAV | NC_005889, | Hu16 | AY530581 | Rh43 | AY530560 |
| | AY388617, | | | | |
| | AAR26465 | | | | |
| AAV11 | AAT46339, | Hu25 | AY530591 | AAV8 | AF513852 |
| | AY631966 | | | | |
| AAV12 | ABI16639, | Hu60 | AY530622 | Rh8 | AY242997 |
| | DQ813647 | | | | |
| Clade A | | Ch5 | AY243021 | Rh1 | AY530556 |
| AAV1 | NC_002077, | Hu3 | AY530595 | Clade F | |
| | AF063497 | | | | |
| AAV6 | NC_001862 | Hu1 | AY530575 | Hu14 | AY530579 |
| | | | | (AAV9) | |
| Hu.48 | AY530611 | Hu4 | AY530602 | Hu31 | AY530596 |
| Hu 43 | AY530606 | Hu2 | AY530585 | Hu32 | AY530597 |
| Hu 44 | AY530607 | Hu61 | AY530623 | HSC1 | MI332400.1 |
| Hu 46 | AY530609 | Clade D | | HSC2 | MI332401.1 |
| Clade B | | Rh62 | AY530573 | HSC3 | MI332402.1 |
| Hu.19 | AY530584 | Rh48 | AY530561 | HSC4 | MI332403.1 |
| Hu.20 | AY530586 | Rh54 | AY530567 | HSC5 | MI332405.1 |
| Hu 23 | AY530589 | Rh55 | AY530568 | HSC6 | MI332404.1 |
| Hu22 | AY530588 | Cy2 | AY243020 | HSC7 | MI332407.1 |
| Hu24 | AY530590 | AAV7 | AF513851 | HSC8 | MI332408.1 |
| Hu21 | AY530587 | Rh35 | AY243000 | HSC9 | MI332409.1 |
| Hu27 | AY530592 | Rh37 | AY242998 | HSC11 | MI332406.1 |
| Hu28 | AY530593 | Rh36 | AY242999 | HSC12 | MI332410.1 |
| Hu 29 | AY530594 | Cy6 | AY243016 | HSC13 | MI332411.1 |
| Hu63 | AY530624 | Cy4 | AY243018 | HSC14 | MI332412.1 |
| Hu64 | AY530625 | Cy3 | AY243019 | HSC15 | MI332413.1 |
| Hu13 | AY530578 | Cy5 | AY243017 | HSC16 | MI332414.1 |
| Hu56 | AY530618 | Rh13 | AY243013 | HSC17 | MI332415.1 |
| Hu57 | AY530619 | Clade E | | Hu68 | |
| Hu49 | AY530612 | Rh38 | AY530558 | Clonal | |
| | | | | Isolate | |
| Hu58 | AY530620 | Hu66 | AY530626 | AAV5 | Y18065, |
| | | | | | AF085716 |
| Hu34 | AY530598 | Hu42 | AY530605 | AAV 3 | NC_001729 |
| Hu35 | AY530599 | Hu67 | AY530627 | AAV 3B | NC_001863 |
| AAV2 | NC_001401 | Hu40 | AY530603 | AAV4 | NC_001829 |
| Hu45 | AY530608 | Hu41 | AY530604 | Rh34 | AY243001 |
| Hu47 | AY530610 | Hu37 | AY530600 | Rh33 | AY243002 |
| Hu51 | AY530613 | Rh40 | AY530559 | Rh32 | AY243003 |
| Hu52 | AY530614 | Rh2 | AY243007 | Others | |
| Hu T41 | AY695378 | Bb1 | AY243023 | Rh74 | |
| Hu S17 | AY695376 | Bb2 | AY243022 | Bearded | |
| | | | | Dragon | |
| | | | | AAV | |
| Hu T88 | AY695375 | Rh10 | AY243015 | Snake | NC_006148.1 |
| | | | | AAV | |
| Hu T71 | AY695374 | Hu17 | AY530582 | | |
| Hu T70 | AY695373 | Hu6 | AY530621 | | |
| Hu T40 | AY695372 | Rh25 | AY530557 | | |
| Hu T32 | AY695371 | Pi2 | AY530554 | | |
| Hu T17 | AY695370 | Pi1 | AY530553 | | |
| Hu LG15 | AY695377 | Pi3 | AY530555 | | |

The terms “viral production cell”, “viral production cell line,” or “viral producer cell” refer to cells used to produce viral vectors. HEK293 and 239T cells are common viral production cell lines. Table 2, below, lists exemplary viral production cell lines for various viral vectors.

TABLE 2
Exemplary viral production cell lines

| | Exemplary Viral |
|---|---|
| Virus Vector | Production Cell Line(s) |
| Adenovirus | HEK293, 911, pTG6559, |
| | PER.C6, GH329, N52.E6, |
| | HeLa-E1, UR, VLI-293 |
| Adeno-Associated Virus | HEK293, Sf9 |
| (AAV) | |
| Retrovirus | HEK293 |
| Lentivirus | 293T |

“HEK293” refers to a cell line originally derived from human embryonic kidney cells 10 grown in tissue culture. The HEK293 cell line grows readily in culture, and is commonly used for viral production. As used herein, “HEK293” may also refer to one or more variant HEK293 cell lines, i.e., cell lines derived from the original HEK293 cell line that additionally comprise one or more genetic alterations. Many variant HEK293 lines have been developed and optimized for one or more particular applications. For example, the 293T cell line contains the SV40 large T-antigen that allows for episomal replication of transfected plasmids containing the SV40 origin of replication, leading to increased expression of desired gene products.

“Sf9” refers to an insect cell line that is a clonal isolate derived from the parental Spodoptera frugiperda cell line IPLB-Sf-21-AE. Sf9 cells can be grown in the absence of serum and can be cultured attached or in suspension.

A “transfection reagent” means a composition that enhances the transfer of nucleic acid into cells. Some transfection reagents commonly used in the art include one or more lipids that bind to nucleic acids and to the cell surface (e.g., Lipofectamine™).

As used herein “sequence identity” refers to the extent to which two optimally aligned polynucleotides or polypeptide sequences are invariant throughout a window of alignment of components, e.g., nucleotides or amino acids. An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. “Percent identity” is the identity fraction times 100. The extent of identity (homology) between two sequences can be ascertained using a computer program and mathematical algorithm. Percentage identity can be calculated using the alignment program Clustal Omega, available at www.ebi.ac.uk/Tools/msa/clustalo using default parameters. See, Sievers et al., “Fast, scalable generation of high-quality protein multiple sequence alignments using Clustal Omega.” (2011 Oct. 11) Molecular systems biology 7:539.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. Therapeutic benefit refers to any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The terms “subject,” “individual,” and “patient” are used interchangeably herein to refer to a vertebrate, such as a mammal. The mammal may be, for example, a mouse, a rat, a rabbit, a cat, a dog, a pig, a sheep, a horse, a non-human primate (e.g., cynomolgus monkey, chimpanzee), or a human. A subject's tissues, cells, or derivatives thereof, obtained in vivo or cultured in vitro are also encompassed. A human subject may be an adult, a teenager, a child (2 years to 14 years of age), an infant (1 month to 24 months), or a neonate (up to 1 month). In some embodiments, the adults are seniors about 65 years or older, or about 60 years or older. In some embodiments, the subject is a pregnant woman or a woman intending to become pregnant.

The term “effective amount” or “therapeutically effective amount” refers to the amount of an agent that is sufficient to achieve an outcome, for example, to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, the term “gene therapy” refers to the process of introducing genetic material into cells to compensate for abnormal genes, or to make a therapeutic protein.

As used herein, “left ventricular ejection fraction” refers to a measurement, expressed as a percentage, of how much blood the left ventricle pumps out with each contraction. For example, an ejection fraction of 60% means that 60% of the total amount of blood in the left ventricle is pushed out with each heartbeat. Left ventricular ejection fraction may be calculated based on the results of tests, such as, echocardiogram, MUGA scan, CAT scan, cardiac catheterization, and nuclear stress test.

As used herein, the term “right ventricular area” refers to measuring the right ventricular area by using long-axis B-mode echocardiography. This measures a 2-dimensional plane of the heart orientated to the long axis.

### AAV Expression Cassettes

The disclosure provides nucleic acid sequences comprising one or more adeno-associated virus (AAV) expression cassettes. In some embodiments, the AAV expression cassette comprises a 5′ inverted terminal repeat (ITR), a promoter, a transgene, and a 3′ ITR. In some embodiments, the transgene is an arrhythmogenic cardiomyopathy-associated gene. In some embodiments, the AAV expression cassette comprises a Kozak sequence, a polyadenylation sequence, and/or a stuffer sequence.

In some embodiments, the AAV expression cassette comprises a nucleic acid sequence of SEQ ID NO: 12, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween). In some embodiments, the AAV expression cassette comprises a nucleic acid sequence of SEQ ID NO: 12.

### (i) Inverted Terminal Repeat

Inverted Terminal Repeat or ITR sequences are sequences that mediate AAV proviral integration and packaging of AAV DNA into virions. ITRs are involved in a variety of activities in the AAV life cycle. For example, the ITR sequences, which can form hairpin structures, play roles in excision from the plasmid, replication of the vector genome and integration and rescue from a host cell genome.

The AAV expression cassettes of the disclosure may comprise a 5′ ITR and a 3′ ITR. The ITR sequences may be about 110 to about 160 nucleotides in length, for example 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159 or 160 nucleotides in length. In some embodiments, the ITR sequences may be about 141 nucleotides in length. In some embodiments, the 5′ ITR is the same length as the 3′ ITR. In some embodiments, the 5′ ITR and the 3′ ITR have different lengths. In some embodiments, the 5′ ITR is longer than the 3′ ITR, and in other embodiments, the 3′ ITR is longer than the 5′ ITR.

The ITRs may be isolated or derived from the genome of any AAV, for example the AAVs listed in Table 1. In some embodiments, at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, at least one of the 5′ ITR and the 3′ITR may be a wild type or mutated ITR isolated or derived from a member of another parvovirus species besides AAV. For example, in some embodiments, an ITR may be a wild type or mutant ITR isolated or derived from bocavirus or parvovirus B19.

In some embodiments, the ITR comprises a modification to promote production of a scAAV. In some embodiments, the modification to promote production of a scAAV is deletion of the terminal resolution sequence (TRS) from the ITR. In some embodiments, the 5′ ITR is a wild type ITR, and the 3′ ITR is a mutated ITR lacking the terminal resolution sequence. In some embodiments, the 3′ ITR is a wild type ITR, and the 5′ ITR is a mutated ITR lacking the terminal resolution sequence. In some embodiments, the terminal resolution sequence is absent from both the 5′ ITR and the 3′ITR. In other embodiments, the modification to promote production of a scAAV is replacement of an ITR with a different hairpin-forming sequence, such as a short hairpin (sh) RNA-forming sequence.

In some embodiments, the 5′ ITR may comprise the sequence of SEQ ID NO: 5, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 3′ ITR may comprise the sequence of SEQ ID NO: 6, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between). In some embodiments, the 5′ ITR comprises the sequence of SEQ ID NO: 5, and the 3′ ITR comprises the sequence of SEQ ID NO: 6.

In some embodiments, the AAV expression cassettes comprise one or more “surrogate” ITRs, i.e., non-ITR sequences that serve the same function as ITRs. See, e.g., Xie, J. et al., Mol. Ther., 25(6): 1363-1374 (2017). In some embodiments, an ITR in an AAV expression cassette is replaced by a surrogate ITR. In some embodiments, the surrogate ITR comprises a hairpin-forming sequence. In some embodiments, the surrogate ITR is a shRNA-forming sequence.

### (ii) Promoters

In some embodiments, the AAV expression cassettes described herein comprise a promoter. In some embodiments, the promoter is a synthetic promoter. In some embodiments, the promoter may comprise a nucleic acid sequence derived from an endogenous promoter and/or an endogenous enhancer.

In some embodiments, the promoter comprises a nucleic acid sequence derived from one or more promoters commonly used in the art for gene expression. For instance, in some embodiments, the promoter further comprises a nucleic acid sequence derived from the CMV promoter, the SV40 early promoter, the SV40 late promoter, the metallothionein promoter, the murine mammary tumor virus (MMTV) promoter, the Rous sarcoma virus (RSV) promoter, the polyhedrin promoter, the chicken β-actin (CBA) promoter, the dihydrofolate reductase (DHFR) promoter, and the phosphoglycerol kinase (PGK) promoter. In some embodiments, the promoter comprises a nucleic acid sequence derived from the chicken β-actin (CBA) promoter, the EF-1 alpha promoter, or the EF-1 alpha short promoter.

In some embodiments, the promoter is capable of expressing the transgene in a cardiac cell. In some embodiments, the promoter is a cell-specific promoter, such as, a cardiac cell-specific promoter. As used herein, a “cell-specific promoter” refers to a promoter that is capable of expressing a transgene at a level that is higher in a particular cell (e.g., cardiac cell), as compared to a control cell (e.g., a non-cardiac cell). Therefore, in some embodiments, the AAV expression cassettes disclosed herein comprise a promoter that expresses the transgene in a cardiac cell at a level that is higher than a level of the transgene expression by the promoter in a non-cardiac cell. In some embodiments, the promoter expresses the transgene in a cardiac cell at a level that is at least about 1.2 fold (for example, about 1.5 fold, about 2 fold, about 2.5 fold, about 3 fold, about 3.5 fold, about 4 fold, about 4.5 fold, about 5 fold, about 5.5 fold, about 6 fold, about 6.5 fold, about 7 fold, about 7.5 fold, about 8 fold, about 8.5 fold, about 9 fold, about 9.5 fold, about 10 fold, about 15 fold, about 20 fold, about 30 fold, about 40 fold, about 50 fold, about 60 fold, about 70 fold, about 80 fold about 90 fold, or about 100 fold, including all values and subranges that lie therebetween) higher than a level of the transgene expression by the promoter in a non-cardiac cell.

In some embodiments, the promoter may comprise a nucleic acid sequence derived from an endogenous promoter and/or an endogenous enhancer, for example, an endogenous promoter and/or an endogenous enhancer of a gene that is expressed at higher levels in cardiac tissue, as compared to non-cardiac tissue. In some embodiments, the promoter is the promoter of the TNNT2 gene encoding cardiac muscle troponin T, and is referred to herein as cardiac troponin T (TNNT2) promoter. In some embodiments, the TNNT2 promoter comprises a nucleic acid sequence derived from: (i) a human TNNT2 promoter, (ii) a chicken TNNT2 promoter, (iii) a mouse TNNT2 promoter, or (iv) any combination thereof. In some embodiments, the TNNT2 promoter comprises a human TNNT2 promoter. In some embodiments, the promoter comprises the sequence of SEQ ID NO: 4, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie there between).

In some embodiments, the AAV expression cassettes described herein further comprise an enhancer. The enhancer may be, for example, the CMV enhancer. In some embodiments, the enhancer comprises the sequence of SEQ ID NO: 16, or a sequence at least 70% identical thereto (for example, at least 75%, at least 80%, at least 85%, at least 90%, at least 95%, at least 96%, at least 97%, at least 98%, at least 99%, at least 99.5%, or 100% identical thereto, inclusive of all values and subranges that lie therebetween).

In some embodiments, the promoter further comprises a nucleic acid sequence derived from any one or more of the following promoters: HMG-COA reductase promoter; sterol regulatory element 1 (SRE-1); phosphoenol pyruvate carboxy kinase (PEPCK) promoter; human C-reactive protein (CRP) promoter; human glucokinase promoter; cholesterol 7-alpha hydroylase (CYP-7) promoter; beta-galactosidase alpha-2,6 sialyltransferase promoter; insulin-like growth factor binding protein (IGFBP-1) promoter; aldolase B promoter; human transferrin promoter; collagen type I promoter; prostatic acid phosphatase (PAP) promoter; prostatic secretory protein of 94 (PSP 94) promoter; prostate specific antigen complex promoter; human glandular kallikrein gene promoter (hgt-1); the myocyte-specific enhancer binding factor MEF-2; muscle creatine kinase promoter; pancreatitis associated protein promoter (PAP); elastase 1 transcriptional enhancer; pancreas specific amylase and elastase enhancer promoter; pancreatic cholesterol esterase gene promoter; uteroglobin promoter; cholesterol side-chain cleavage (SCC) promoter; gamma-gamma enolase (neuron-specific enolase, NSE) promoter; neurofilament heavy chain (NF-H) promoter; human CGL-1/granzyme B promoter; the terminal deoxy transferase (TdT), lambda 5, VpreB, and lck (lymphocyte specific tyrosine protein kinase p561ck) promoter; the humans CD2 promoter and its 3′ transcriptional enhancer; the human NK and T cell specific activation (NKG5) promoter; pp60c-src tyrosine kinase promoter; organ-specific neoantigens (OSNs), mw 40 kDa (p40) promoter; colon specific antigen-P promoter; human alpha-lactalbumin promoter; phosphoeholpyruvate carboxykinase (PEPCK) promoter, HER2/neu promoter, casein promoter, IgG promoter, Chorionic Embryonic Antigen promoter, elastase promoter, porphobilinogen deaminase promoter, insulin promoter, growth hormone factor promoter, tyrosine hydroxylase promoter, albumin promoter, alphafetoprotein promoter, acetyl-choline receptor promoter, alcohol dehydrogenase promoter, alpha or beta globin promoter, T-cell receptor promoter, the osteocalcin promoter the IL-2 promoter, IL-2 receptor promoter, whey (wap) promoter, and the MHC Class II promoter. In some embodiments, the AAV expression cassettes disclosed herein further comprise a nucleic acid sequence derived from any one or more of the promoters, enhancers and/or other sequences described in U.S. Pat. No. 8,708,948B2, U.S. Pat. No. 9,1385,96B2, U.S. Pat. No. 10,286,085B2, and U.S. Patent No. U.S. Pat. No. 8,538,520B2, the contents of each of which are incorporated herein by reference in their entireties.

(iii) Arrhythmogenic Cardiomyopathy-Associated Gene

As used herein, an “arrhythmogenic cardiomyopathy-associated gene” refers to any gene in a subject with arrhythmogenic cardiomyopathy which can be targeted by gene therapy to alleviate at least one symptom of arrhythmogenic cardiomyopathy. In some embodiments, the level of the protein encoded by the arrhythmogenic cardiomyopathy-associated gene is reduced or undetectable in subjects with arrhythmogenic cardiomyopathy. In some embodiments, the arrhythmogenic cardiomyopathy-associated gene encodes a protein that contributes to normal heart function. In some embodiments, the arrhythmogenic cardiomyopathy-associated gene encodes a protein that contributes to normal myocardial function. In some embodiments, the arrhythmogenic cardiomyopathy-associated gene encodes a protein that contributes to normal function of desmosomes in cells that contain desmosomes, such as, cardiac cells and epithelial cells.

In some embodiments, mutations in the arrhythmogenic cardiomyopathy-associated gene (e.g., PKP2 gene) is present in subjects with arrhythmogenic cardiomyopathy. In some embodiments, loss of function or haploinsufficiency of the arrhythmogenic cardiomyopathy-associated gene (e.g., PKP2 gene) is present in subjects with arrhythmogenic cardiomyopathy. In some embodiments, mutations in the arrhythmogenic cardiomyopathy-associated gene, or loss of function of the arrhythmogenic cardiomyopathy-associated gene, is associated with, promotes or causes arrhythmogenic cardiomyopathy. The type of mutation in the arrhythmogenic cardiomyopathy-associated gene (e.g., PKP2 gene) is not limited, and may be an insertion, deletion, duplication and/or substitution. In some embodiments, the mutation in the PKP2 gene is any PKP2 mutation that has been identified in patients with arrhythmogenic cardiomyopathy. For instance, the mutation in the PKP2 gene is selected from one or more PKP2 gene mutations described in Gerull B, et al. Mutations in the desmosomal protein plakophilin-2 are common in arrhythmogenic right ventricular cardiomyopathy. Nat Genet. 2004 November; 36(11):1162-4, which is incorporated herein by reference in its entirety for all purposes.

The disclosure provides AAV expression cassettes comprising an arrhythmogenic cardiomyopathy-associated gene. In some embodiments, an AAV expression cassette comprises an arrhythmogenic cardiomyopathy-associated gene which encodes a protein, including therapeutic (e.g., for medical or veterinary uses) or immunogenic (e.g., for vaccines) polypeptide. In some embodiments, the AAV expression cassette comprises a mammalian arrhythmogenic cardiomyopathy-associated gene. In some embodiments, the AAV expression cassette comprises a human arrhythmogenic cardiomyopathy-associated gene. In some embodiments, the AAV expression cassette comprises an arrhythmogenic cardiomyopathy-associated gene that encodes plakophilin-2.

In some embodiments, the transgene encodes a human plakophilin-2. In some embodiments, the transgene encodes an isoform 2a of human plakophilin-2. In some embodiments, the isoform 2a of human plakophilin-2 comprises the amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 1. In some embodiments, the isoform 2a of human plakophilin-2 comprises the amino acid sequence of SEQ ID NO: 1, or a sequence at least 90% identical thereto.

In some embodiments, the transgene encodes an RNA transcript variant 2a of the PKP2 gene. In some embodiments, the transgene comprises the nucleic acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 2. In some embodiments, the transgene comprises the nucleic acid sequence of SEQ ID NO: 2, or a sequence at least 90% identical thereto.

In some embodiments, the transgene encodes an isoform 2b of human plakophilin-2. In some embodiments, the isoform 2b of human plakophilin-2 comprises an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 3. In some embodiments, the isoform 2b of human plakophilin-2 comprises the amino acid sequence of SEQ ID NO: 3, or a sequence at least 90% identical thereto. In some embodiments, the transgene encodes an RNA transcript variant 2b of the PKP2 gene. In some embodiments, the PKP2 gene is a human PKP2 gene.

In some embodiments, the AAV expression cassette comprises a Kozak sequence. The Kozak sequence is a nucleic acid sequence that functions as a protein translation initiation site in many eukaryotic mRNA transcripts. In some embodiments, the Kozak sequence overlaps with the start codon. In some embodiments, the Kozak sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 10 or acagccacc. In some embodiments, the Kozak sequence comprises a nucleic acid sequence of SEQ ID NO: 10, or a sequence at least 90% identical thereto; or a nucleic acid sequence of acagccacc, or a sequence at least 90% identical thereto.

### (iv) Polyadenylation (PolyA) Signal

Polyadenylation signals are nucleotide sequences found in nearly all mammalian genes and control the addition of a string of approximately 200 adenosine residues (the poly(A) tail) to the 3′ end of the gene transcript. The poly(A) tail contributes to mRNA stability, and mRNAs lacking the poly(A) tail are rapidly degraded. There is also evidence that the presence of the poly(A) tail positively contributes to the translatability of mRNA by affecting the initiation of translation.

In some embodiments, the AAV expression cassettes of the disclosure comprise a polyadenylation signal. The polyadenylation signal may be selected from the polyadenylation signal of simian virus 40 (SV40), rabbit beta globin (rBG), α-globin, β-globin, human collagen, human growth hormone (hGH), polyoma virus, human growth hormone (hGH) and bovine growth hormone (bGH).

In some embodiments, the AAV expression cassette comprises a bGH polyadenylation signal. In some embodiments, the bGH polyadenylation signal comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 8. In some embodiments, the bGH polyadenylation signal comprises a nucleic acid sequence of SEQ ID NO: 8, or a sequence at least 90% identical thereto.

In some embodiments, the polyadenylation signal is the SV40 polyadenylation signal. In some embodiments, the polyadenylation signal is the rBG polyadenylation signal. In some embodiments, the polyadenylation signal comprises the sequence of SEQ ID NO: 17 or SEQ ID NO: 18. In some embodiments, the polyadenylation signal comprises a sequence at least 90%, at least 95%, at least 96%, at least 97%, at least 98%, or at least 99% identical to the sequence of SEQ ID NO: 17 or SEQ ID NO: 18.

### (v) Stuffer Sequences

AAV vectors typically accept inserts of DNA having a defined size range which is generally about 4 kb to about 5.2 kb, or slightly more. Thus, for shorter sequences, it may be necessary to include additional nucleic acid in the insert fragment to achieve the required length which is acceptable for the AAV vector. Accordingly, in some embodiments, the AAV expression cassettes of the disclosure may comprise a stuffer sequence. The stuffer sequence may be for example, a sequence between 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, or 4,500-5,000, or more nucleotides in length. The stuffer sequence can be located in the cassette at any desired position such that it does not prevent a function or activity of the vector.

In some embodiments, the AAV cassette comprises at least one stuffer sequence. In some embodiments, the stuffer sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 9. In some embodiments, the stuffer sequence comprises a nucleic acid sequence of SEQ ID NO: 9, or a sequence at least 90% identical thereto. In some embodiments, the stuffer sequence comprises a nucleic acid sequence of SEQ ID NO: 9, or a portion thereof. In some embodiments, the stuffer sequence comprises a portion (e.g., a 500-nucleotide long portion) of the nucleic acid sequence of SEQ ID NO: 9, or a sequence at least 90% identical thereto.

### (vi) Intronic Sequences

In some embodiments, the AAV expression cassettes of the disclosure may comprise an intronic sequence. In some embodiments, inclusion of an intronic sequence enhances expression compared with expression in the absence of the intronic sequence.

In some embodiments, the intronic sequence is a hybrid or chimeric sequence. In some embodiments, the intronic sequence is isolated or derived from an intronic sequence of one or more of SV40, β-globin, chicken beta-actin, minute virus of mice (MVM), factor IX, and/or human IgG (heavy or light chain). In some embodiments, the intronic sequence is chimeric. In some embodiments, the intronic sequence comprises a nucleic acid sequence having at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to the nucleic acid sequence of SEQ ID NO: 7. In some embodiments, the intronic sequence comprises the sequence of SEQ ID NO: 7, or a sequence at least 90% identical thereto. In some embodiments, the intronic sequence comprises the sequence of SEQ ID NO: 7.

### AAV Production Methods

The AAV expression cassettes described herein may be incorporated into a vector (e.g., a plasmid or a bacmid) using standard molecular biology techniques. The disclosure provides vectors comprising any one of the AAV expression cassettes described herein. The vector (e.g., plasmid or bacmid) may further comprise one or more genetic elements used during production of AAV, including, for example, AAV rep and cap genes, and helper virus protein sequences.

The AAV expression cassettes, and vectors (e.g., plasmids) comprising the AAV expression cassettes described herein may be used to produce recombinant AAV vectors.

The disclosure provides methods for producing a recombinant AAV vector comprising contacting an AAV producer cell (e.g., an HEK293 cell) with an AAV expression cassette, or vector (e.g., plasmid) of the disclosure. The disclosure further provides cells comprising any one of the AAV expression cassettes, or vectors disclosed herein. In some embodiments, the method further comprises contacting the AAV producer cell with one or more additional plasmids encoding, for example, AAV rep and cap genes, and helper virus protein sequences. In some embodiments, a method for producing a recombinant AAV vector comprises contacting an AAV producer cell (e.g., an insect cell such as a Sf9 cell) with at least one insect cell-compatible vector comprising an AAV expression cassette of the disclosure. An “insect cell-compatible vector” is any compound or formulation (biological or chemical), which facilitates transformation or transfection of an insect cell with a nucleic acid. In some embodiments, the insect cell-compatible vector is a baculoviral vector. In some embodiments, the method further comprises maintaining the insect cell under conditions such that AAV is produced.

The disclosure provides recombinant AAV vectors produced using any one of the methods disclosed herein. The recombinant AAV vectors produced may be of any serotype, for example AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the recombinant AAV vectors produced may comprise one or more amino acid modifications (e.g., substitutions and/or deletions) compared to the native AAV capsid. For example, the recombinant AAV vectors may be modified AAV vectors derived from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV. In some embodiments, the recombinant AAV vector is a single-stranded AAV (ssAAV). In some embodiments, the recombinant AAV vector is a self-complementary AAV (scAAV).

In some embodiments, the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV. In some embodiments, the AAV vector comprises a capsid protein with one or more substitutions or mutations, as compared to a wild type AAV capsid protein. The recombinant AAV vectors disclosed herein may be used to transduce target cells with the transgene sequence, for example by contacting the recombinant AAV vector with a target cell.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 13. In some embodiments, In some embodiments, the AAV vector comprises a capsid protein comprising the amino acid sequence of SEQ ID NO: 13, or a sequence at least 90% identical thereto. In some embodiments, the AAV vector comprises a capsid protein comprising the amino acid sequence of SEQ ID NO: 13.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 14. In some embodiments, the AAV vector comprises a capsid protein comprising the amino acid sequence of SEQ ID NO: 14, or a sequence at least 90% identical thereto. In some embodiments, the AAV vector comprises a capsid protein comprising the amino acid sequence of SEQ ID NO: 14.

In some embodiments, the AAV vector comprises a capsid protein comprising: an amino acid sequence with at least 70% identity (for example, at least 75%, at least 80%, at least 85%, at least 90%, at least 95%, at least 96%, at least 97%, at least 98%, at least 99%, at least 99.5%, or 100% identity, inclusive of all values and subranges that lie therebetween) to SEQ ID NO: 15. In some embodiments, the AAV vector comprises a capsid protein comprising the amino acid sequence of SEQ ID NO: 15, or a sequence at least 90% identical thereto. In some embodiments, the AAV vector comprises a capsid protein comprising the amino acid sequence of SEQ ID NO: 15.

In some embodiments, the AAV vector comprises a capsid protein comprising: (i) the amino acid sequence of SEQ ID NO: 13, or a sequence at least 90% identical thereto, or (ii) the amino acid sequence of SEQ ID NO: 14, or a sequence at least 90% identical thereto, or (iii) the amino acid sequence of SEQ ID NO: 15, or a sequence at least 90% identical thereto.

### Methods of Expression and Treatment

The disclosure provides compositions comprising any one of the nucleic acids, AAV expression cassettes, plasmids, cells, or recombinant AAV vectors disclosed herein. In some embodiments, the compositions disclosed herein comprise at least one pharmaceutically acceptable carrier, excipient, and/or vehicle, for example, solvents, buffers, solutions, dispersion media, coatings, antibacterial and antifungal agents, isotonic and absorption delaying agents. In some embodiments, the pharmaceutically acceptable carrier, excipient, and/or vehicle may comprise saline, buffered saline, dextrose, water, glycerol, sterile isotonic aqueous buffer, and combinations thereof. In some embodiments, the pharmaceutically acceptable carrier, excipient, and/or vehicle comprises phosphate buffered saline, sterile saline, lactose, sucrose, calcium phosphate, dextran, agar, pectin, peanut oil, sesame oil, pharmaceutical grades of mannitol, lactose, starch, magnesium stearate, sodium saccharine, cellulose, magnesium carbonate, polyol (e.g., glycerol, propylene glycol, and liquid polyethylene glycol, and the like) or suitable mixtures thereof. In some embodiments, the compositions disclosed herein further comprise minor amounts of emulsifying or wetting agents, or pH buffering agents.

In some embodiments, the compositions disclosed herein further comprise other conventional pharmaceutical ingredients, such as preservatives, or chemical stabilizers, such as chlorobutanol, potassium sorbate, sorbic acid, sulfur dioxide, propyl gallate, the parabens, ethyl vanillin, glycerin, phenol, parachlorophenol or albumin. In some embodiments, the compositions disclosed herein may further comprise antibacterial and antifungal agents, such as, parabens, chlorobutanol, phenol, sorbic acid or thimerosal; isotonic agents, such as, sugars or sodium chloride and/or agents delaying absorption, such as, aluminum monostearate and gelatin.

The disclosure provides methods of expressing an arrhythmogenic cardiomyopathy-associated transgene in a cell, comprising: contacting the cell with any one of the nucleic acid molecules, plasmids, cells, recombinant AAV vectors, or compositions disclosed herein, thereby expressing the arrhythmogenic cardiomyopathy-associated transgene in the cell.

The disclosure provides methods of expressing an arrhythmogenic cardiomyopathy-associated transgene in a tissue, comprising: contacting the tissue with any one of the nucleic acid molecules, plasmids, cells, recombinant AAV vectors, or compositions disclosed herein, thereby expressing the arrhythmogenic cardiomyopathy-associated transgene in the tissue. In some embodiments, the tissue comprises at least one cell, and at least one desmosomal junction.

In some embodiments, the cell is a cardiac cell, an endothelial cell, a skin cell, a bladder cell, or a gastrointestinal mucosal cell. In some embodiments, the cell is a cardiac cell. In some embodiments, the cell is a dividing cell, such as a cultured cell in cell culture. In some embodiments, the cell is a non-dividing cell. In some embodiments, the arrhythmogenic cardiomyopathy-associated gene is delivered to the cell in vitro, e.g., to produce the arrhythmogenic cardiomyopathy-associated polypeptide in vitro or for ex vivo gene therapy.

In some embodiments, the contacting step is performed in vitro, ex vivo, or in vivo. In some embodiments, the contacting step is performed in vivo in a subject in need thereof. In some embodiments, the contacting step comprises administering a therapeutically effective amount of the nucleic acid molecule, the plasmid, the recombinant AAV vector, or the composition to the subject. In some embodiments, the subject suffers from, or is at a risk of developing the arrhythmogenic cardiomyopathy.

The disclosure provides methods for treating arrhythmogenic cardiomyopathy in a subject in need thereof, comprising administering to the subject a therapeutically effective amount of any one of the nucleic acid molecules, plasmids, cells, recombinant AAV vectors, or compositions disclosed herein, thereby treating arrhythmogenic cardiomyopathy in the subject. In some embodiments, the subject suffers from, or is at a risk of developing the arrhythmogenic cardiomyopathy. In some embodiments, the arrhythmogenic cardiomyopathy is arrhythmogenic right ventricular cardiomyopathy. In some embodiments, the arrhythmogenic cardiomyopathy is associated with, promoted by, or caused by a genetic change. In some embodiments, the genetic change comprises one or more genetic changes (for example, one or more deletions, insertions, duplications and/or substitutions) to the PKP2 gene, as compared to the wild type PKP2 gene, and/or alterations to the expression and/or activity of the PKP2 protein, as compared with a wild type PKP2 protein. In some embodiments, the mutation in the PKP2 gene results in PKP2 haploinsufficiency. In some embodiments, the subject at a risk of developing arrhythmogenic cardiomyopathy is a newborn who is identified as carrying a mutation in the PKP2 gene. In some embodiments, the arrhythmogenic cardiomyopathy-associated gene (e.g., PKP2) is targeted by gene therapy to increase its expression and/or function.

In some embodiments, the method comprises diminishing the severity of; delaying the onset or progression of, and/or eliminating a symptom of the arrhythmogenic cardiomyopathy. In some embodiments, the symptom of the arrhythmogenic cardiomyopathy comprises: (a) re-entrant ventricular tachycardia, (b) syncope, (c) sudden death, or (d) any combination thereof. In some embodiments, the arrhythmogenic cardiomyopathy is associated with: (a) decreased mechanical stability between cardiomyocytes of the subject, (b) disruption of gap junctions in the cardiac tissue of the subject, (c) decreased sodium currents in the cardiac tissue of the subject, (d) fibrosis of the right ventricular myocardium, or (e) any combination thereof.

In some embodiments, the method comprises increasing the mechanical stability between cardiomyocytes of the subject. In some embodiments, the method comprises improving the function of gap junctions in the cardiac tissue of the subject. In some embodiments, the method comprises increasing sodium currents in the cardiac tissue of the subject. In some embodiments, the method comprises decreasing fibrosis of the right ventricular myocardium of the subject.

In some embodiments, the methods comprise increasing the left ventricular ejection fraction of the heart, as compared to a control subject having arrhythmogenic cardiomyopathy, wherein the control subject has not been administered the therapeutically effective amount. In some embodiments, the methods comprise increasing the left ventricular ejection fraction of the heart, as compared to the left ventricular ejection fraction of the heart of the subject prior to administration of the therapeutically effective amount. In some embodiments, the methods comprise increasing the left ventricular ejection fraction of the heart to a value in the range of about 30% to about 80%, for example, about 35%, about 40%, about 50%, about 55%, about 60%, about 65%, about 70%, about 75%, or about 80%, including the subranges and values that lie therebetween. In some embodiments, the methods comprise increasing the left ventricular ejection fraction of the heart to about 60%.

In some embodiments, the methods comprise decreasing the right ventricular area of the heart, as compared to a control subject having arrhythmogenic cardiomyopathy, wherein the control subject has not been administered the therapeutically effective amount. In some embodiments, the methods comprise decreasing the right ventricular area of the heart, as compared to the right ventricular area of the heart of the subject prior to administration of the therapeutically effective amount.

In some embodiments, the methods comprise prolonging the survival of the subject, as compared to a control subject having arrhythmogenic cardiomyopathy, wherein the control subject has not been administered the therapeutically effective amount. In some embodiments, the methods comprise prolonging the survival of the subject, as compared to the expected survival of the subject prior to administration of the therapeutically effective amount. In some embodiments, the methods comprise prolonging the survival of the subject by a value in the range of about 3 months to about 50 years (for example, about 6 months, about 1 year, about 5 years, about 10 years, about 15 years, about 20 years, about 25 years, about 30 years, about 35 years, about 40 years, about 45 years, about 50 years, including the subranges and values that lie therebetween), as compared to: (i) a control subject having arrhythmogenic cardiomyopathy, wherein the control subject has not been administered the therapeutically effective amount, or (ii) the expected survival of the subject prior to administration of the therapeutically effective amount. Dosages of the recombinant AAV vector to be administered to a subject depend upon the mode of administration, the disease or condition to be treated and/or prevented, the individual subject's condition, the particular virus vector or capsid, the nucleic acid to be delivered, and the like, and can be determined in a routine manner. Exemplary doses for achieving therapeutic effects are titers of at least about 10⁵, about 10⁶, about 10⁷, about 10⁸, about 10⁹, about 10¹⁰, about 10¹¹, about 10¹², about 10¹³, about 10¹⁴, about 10¹⁵ transducing units, optionally about 10⁸ to about 10¹³ transducing units.

In some embodiments, the methods comprise increasing the ejection fraction of the subject, as compared to a control subject having arrhythmogenic cardiomyopathy, wherein the control subject has not been administered the therapeutically effective amount. In some embodiments, the methods comprise increasing the ejection fraction of the subject, as compared to the ejection fraction of the subject prior to administration of the therapeutically effective amount.

In some embodiments, the methods comprise increasing the stroke volume of the subject, as compared to a control subject having arrhythmogenic cardiomyopathy, wherein the control subject has not been administered the therapeutically effective amount. In some embodiments, the methods comprise increasing the stroke volume of the subject, as compared to the stroke volume of the subject prior to administration of the therapeutically effective amount.

In some embodiments, the methods comprise increasing the cardiac output of the subject, as compared to a control subject having arrhythmogenic cardiomyopathy, wherein the control subject has not been administered the therapeutically effective amount. In some embodiments, the methods comprise increasing the cardiac output of the subject, as compared to the cardiac output of the subject prior to administration of the therapeutically effective amount.

In some embodiments, the methods comprise increasing the percent fractional shortening of the subject, as compared to a control subject having arrhythmogenic cardiomyopathy, wherein the control subject has not been administered the therapeutically effective amount. In some embodiments, the methods comprise increasing the percent fractional shortening of the subject, as compared to the percent fractional shortening of the subject prior to administration of the therapeutically effective amount.

In some embodiments, the methods comprise increasing the left ventricular outflow tract velocity time integral of the subject, as compared to a control subject having arrhythmogenic cardiomyopathy, wherein the control subject has not been administered the therapeutically effective amount. In some embodiments, the methods comprise increasing the left ventricular outflow tract velocity time integral of the subject, as compared to the left ventricular outflow tract velocity time integral of the subject prior to administration of the therapeutically effective amount.

In some embodiments, the methods comprise decreasing the left ventricular volume of the subject, as compared to a control subject having arrhythmogenic cardiomyopathy, wherein the control subject has not been administered the therapeutically effective amount. In some embodiments, the methods decreasing the left ventricular volume of the subject, as compared to the left ventricular volume of the subject prior to administration of the therapeutically effective amount.

In particular embodiments, more than one administration (e.g., two, three, four or more administrations) may be employed to achieve the desired level of gene expression over a period of various intervals, e.g., daily, weekly, monthly, yearly, etc.

In some embodiments, the subject is a human subject. In some embodiments, the nucleic acid molecule, the plasmid, the cell, the recombinant AAV vector, or composition is administered to the cardiac tissue of the subject. In some embodiments, the nucleic acid molecule, the plasmid, the cell, the recombinant AAV vector, or composition is administered to the left atrium, right atrium, left ventricle, right ventricle and/or septum. In some embodiments, the nucleic acid molecule, the plasmid, the cell, the recombinant AAV vector, or composition is administered to the subject, via intravenous administration, intra-arterial administration, intra-aortic administration, direct cardiac injection, coronary artery perfusion, or any combination thereof.

Other modes of administration include oral, transmucosal, intrathecal, transdermal, parenteral (e.g., intravenous, subcutaneous, intradermal, intramuscular [including administration to skeletal, diaphragm and/or cardiac muscle], intradermal, intrapleural, intracerebral, and intraarticular), intralymphatic, and the like, as well as direct tissue or organ injection (e.g., to liver, skeletal muscle, cardiac muscle, diaphragm muscle, or brain). Delivery to a target tissue can also be achieved by delivering a depot comprising the virus vector and/or capsid. In representative embodiments, a depot comprising the virus vector and/or capsid is implanted into skeletal, cardiac and/or diaphragm muscle tissue or the tissue can be contacted with a film or other matrix comprising the virus vector and/or capsid.

In some embodiments, the methods disclosed herein may comprise administering to the subject a therapeutically effective amount of any one of the nucleic acids, AAV expression cassettes, plasmids, cells, recombinant AAV vectors, or compositions disclosed herein in combination with one or more secondary therapies targeting arrhythmogenic cardiomyopathy. In some embodiments, the methods of treating and/or delaying the onset of at least one symptom of arrhythmogenic cardiomyopathy in a subject disclosed herein may further comprise administering one or more secondary therapies targeting arrhythmogenic cardiomyopathy. In some embodiments, the secondary therapy comprises: administration of a drug, such as a beta blocker or amiodarone, implantable cardioverter defibrillators (ICDs), catheter ablation, or any combination thereof. Non-limiting examples of beta blockers include acebutolol, atenolol, bisoprolol, metoprolol, nadolol, nebivolol, and propranolol.

The term administered “in combination,” as used herein, is understood to mean that two (or more) different treatments are delivered to the subject during the course of the subject's affliction with the disorder (e.g., arrhythmogenic cardiomyopathy), such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent” delivery. In other embodiments, the delivery of one treatment ends before the delivery of the other treatment begins, which may be referred to as “sequential” delivery.

In some embodiments, the treatment is more effective because of combined administration. For example, the second treatment is more effective, an equivalent effect is seen with less of the second treatment, or the second treatment reduces symptoms to a greater extent than would be seen if the second treatment were administered in the absence of the first treatment, or the analogous situation is seen with the first treatment. The effect of the two treatments can be partially additive, wholly additive, or greater than additive (synergistic).

All papers, publications and patents cited in this specification are herein incorporated by reference as if each individual paper, publication, or patent were specifically and individually indicated to be incorporated by reference and are incorporated herein by reference to disclose and describe the methods and/or materials in connection with which the publications are cited. However, mention of any reference, article, publication, patent, patent publication, and patent application cited herein is not, and should not be taken as an acknowledgment or any form of suggestion that they constitute valid prior art or form part of the common general knowledge in any country in the world.

Unless the context indicates otherwise, it is specifically intended that the various features described herein can be used in any combination.

Unless otherwise defined, all technical and scientific terms used herein have the same meaning as commonly understood by one of ordinary skill in the art to which this disclosure belongs.

It is to be understood that the description above as well as the examples that follow are intended to illustrate, and not limit, the scope of the invention. Other aspects, advantages, and modifications within the scope of the invention will be apparent to those skilled in the art to which the invention pertains.

### EXAMPLES

The following examples, which are included herein for illustration purposes only, are not intended to be limiting.

### Example 1: Preparation of AAV Expression Cassettes and Recombinant AAV Vectors in Mammalian Cells

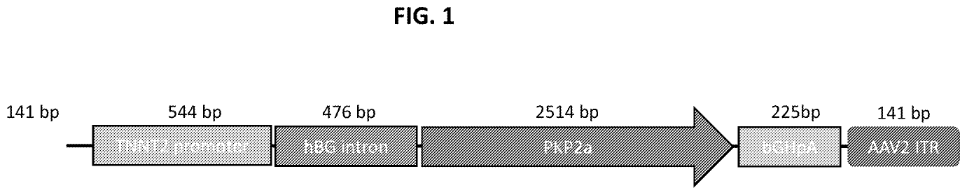

An AAV expression cassette comprising the following elements: (i) a 5′ AAV ITR of SEQ ID NO: 5, (ii) a cardiac troponin T (TNNT2) promoter of SEQ ID NO: 4; (iii) a human beta-globin (hBG) intron of SEQ ID NO: 7; (iv) a human PKP2a transgene sequence of SEQ ID NO: 2; (v) a bovine growth hormone (bGH) polyA tail of SEQ ID NO: 8; and (vi) a 3′ AAV ITR of SEQ ID NO: 6 was generated using standard cloning techniques. See FIG. 1 showing a schematic representation of the AAV cassette. The AAV cassette comprises the sequence of SEQ ID NO: 12.

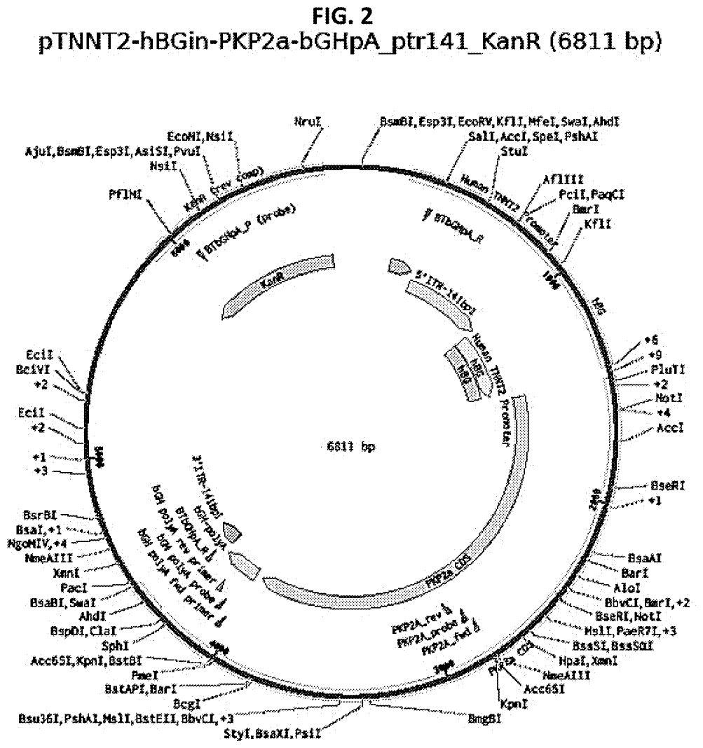

The AAV expression cassette was incorporated into a plasmid (FIG. 2) and transfected into viral production cells (e.g., HEK293) using an appropriate transfection reagent (e.g., Lipofectamine™), along with a Rep/Cap plasmid encoding the Rep and Cap genes, and a helper plasmid comprising various helper sequences required for AAV production (E4, E2a and VA). After incubation at 37° C. for a predetermined period of time, AAV particles were collected from the media or the cells were lysed to release the AAV particles. The AAV particles were then purified, titered, and stored at −80° C. for later use.

In the manner described above, AAV particles comprising the disclosed AAV expression cassette of SEQ ID NO: 12 and a capsid protein having the amino acid sequence of SEQ ID NO: 13 were generated—these AAV particles are referred to herein as “AAV.STRV47-hTNNT2.PKP2a”.

Similarly, AAV particles comprising the disclosed AAV expression cassette of SEQ ID NO: 12 and a capsid protein having the amino acid sequence of SEQ ID NO: 14 (referred to herein as “AAV.STRV5-hTNNT2.PKP2a” were generated.

Finally, AAV particles comprising the disclosed AAV expression cassette of SEQ ID NO: 12 and a capsid protein having the amino acid sequence of SEQ ID NO: 15 (referred to herein as “AAV.STRV84-hTNNT2.PKP2a” were also generated.

### Example 2: Increased PKP2 mRNA and Protein Levels in Cardiomyocytes Upon Treatment with AAV.STRV47-hTNNT2.PKP2a

Healthy normal human iPSC-derived cardiomyocytes were treated with a 50K MOI of AAV.STRV47-hTNNT2.PKP2a 3 days after plating. PKP2a mRNA and PKP2a protein expression were assessed via RT-qPCR and Western Blot, respectively, 7 days after treatment. 1 μM of an antisense oligonucleotide (ASO) against PKP2 was used to knockdown (KD) PKP2 as a control. For mRNA expression, RNA was collected using cells-to-CT kits following the manufacturer's protocol (Invitrogen, Cat #A35377). RT-qPCR was run to measure mRNA expression using TaqMan probes against PKP2 (Hs00428040_m1) and the housekeeping gene UBC (Hs00824723_m1).

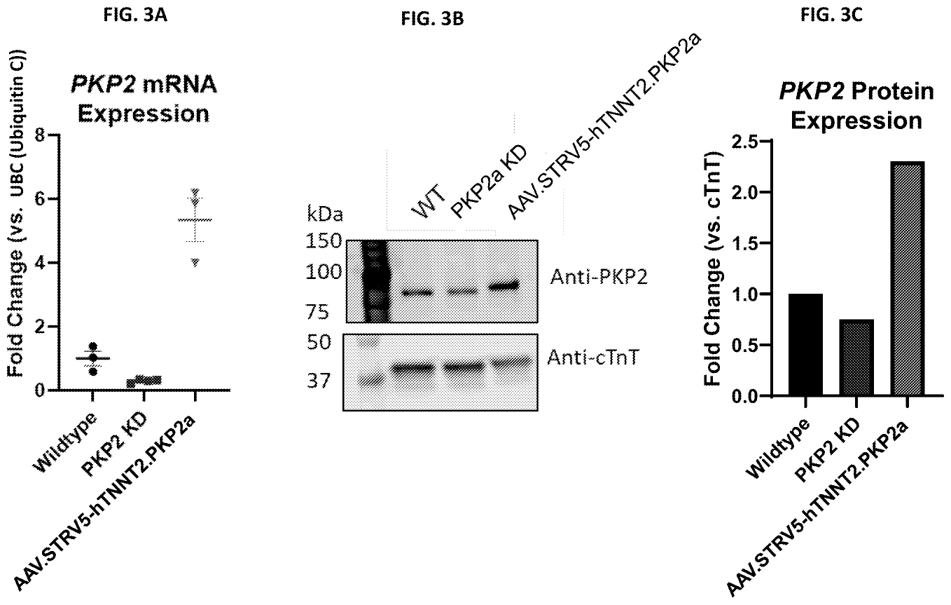

As shown in FIG. 3A, the results show that the PKP2 mRNA expression is reduced upon knockdown of PKP2 using the antisense oligonucleotide. However, PKP2 mRNA was increased several-fold as compared to wild type cells upon transduction of cells with AAV.STRV47-hTNNT2.PKP2a. These results indicate that the treatment of cardiomyocytes with AAV.STRV47-hTNNT2.PKP2a results in increased expression of PKP2 mRNA.

For protein expression, plates were lysed with 300 ul 1×RIPA plus protease inhibitors. Plates were scraped and then rocked for 15 minutes in the cold room. The cell lysate was collected and centrifuged at 4° C. for 10 minutes. The supernatant was collected. Protein lysates were diluted with 4×LiCOR sample buffer and samples were loaded on a Tris/Glycine 20-4% SDS-page gel and subjected to electrophoresis until the dye front reached the bottom of the gel. The gel was transferred to PDVF using the multiMW setting on the BioRad Trans-Blot Turbo system following the manufacturer's protocol. Membranes were blocked for 30 minutes at room temperature with LiCOR blocking buffer then incubated with antibodies against PKP2 (American Research Products, Inc.), cTnT (Abcam ab45932), or beta-tubulin at 1:1000 dilution.

As shown in FIG. 3B and as quantified in FIG. 3C, the results indicate that PKP2 protein expression is reduced upon knockdown of PKP2 using antisense oligonucleotide. However, the PKP2 protein was clearly increased, as compared to wild type cells, upon transduction of cells with the AAV.STRV47-hTNNT2.PKP2a. These results indicate that the treatment of cardiomyocytes with AAV.STRV47-hTNNT2.PKP2a results in increased expression of PKP2 protein.

### Example 3: AAV.STRV5-hTNNT2.PKP2a can Rescue PKP2a Expression Levels in an iPSC PKP2-KO Cardiomyocyte Model

Human PKP1 knockout iPSC cells were generated by CRISPR/Cas9 mediated homozygous insertion of a premature stop codon (S70X). The cells were then differentiated into cardiomyocytes. Methods of generation of cardiomyocytes are described in Burridge et al., 2014, Nat Methods. 2014 August; 11(8): 855-60; and Lian et al., 2012, Proc Natl Acad Sci USA. 2012 Jul. 3; 109(27): E1848-57, the contents of each of which are incorporated herein by reference in their entireties. Successful differentiation resulted in expression of the cardiomyocyte marker TNNT2 and spontaneously beating cells.

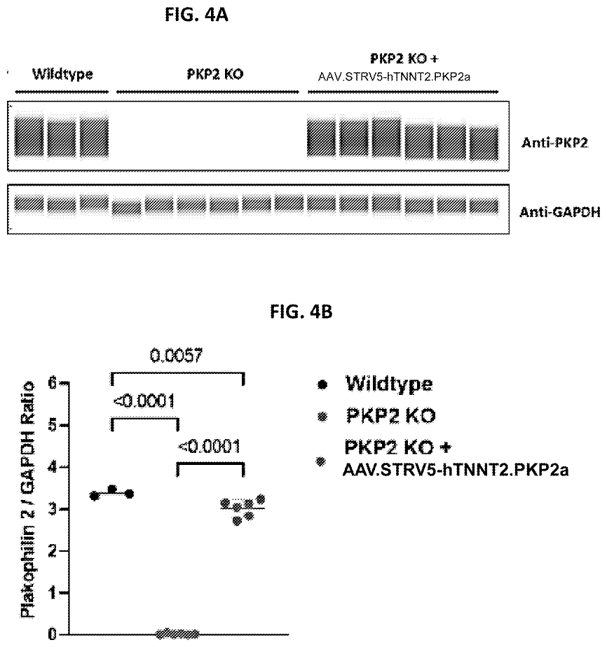

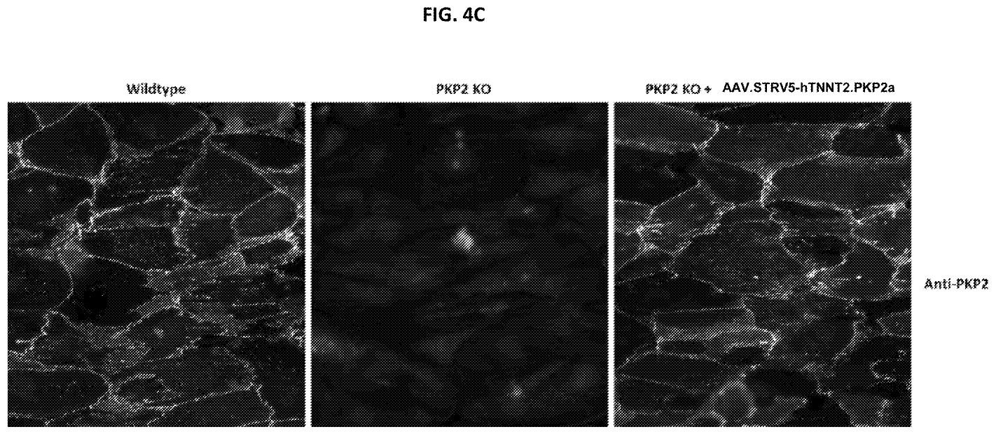

Notably, transduction of iPSC PKP2-KO cardiomyocytes with AAV.STRV5-hTNNT2.PKP2a resulted in PKP2a expression being restored to wild type levels 10 days after transduction. (FIGS. 4A and 4B). PKP2a expression was then examined by immunofluorescence, and correct plasma membrane localization of the PKP2a expressed using AAVs was observed in transduced iPSC PKP2-KO cardiomyocytes resembling that of wild type. FIG. 4C.

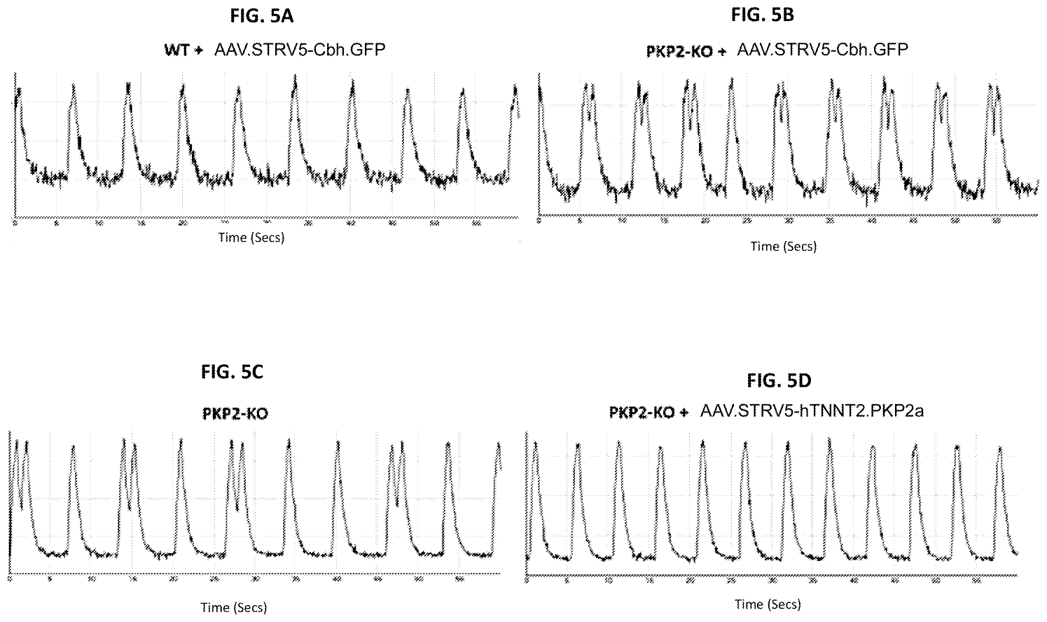

Knocking out the PKP2a protein also impacts the electrical phenotype of human iPSC cardiomyocytes. Calcium transients were measured in spontaneously beating confluent cardiomyocyte cultures. A transduction control (encoding GFP) was included to account for any impact of transduction. Calcium recordings in PKP2-KO cardiomyocytes showed early after transients (dual peaks seen in FIGS. 5B and 5C), which represents the type of dysfunctional calcium handling that could underlie arrhythmias. In iPSC PKP2-KO cardiomyocytes that were transduced with AAV.STRV5-hTNNT2.PKP2a, the restoration of PKP2a expression described above, resulted in normal calcium transients resembling those seen in WT iPSC cardiomyocytes (FIGS. 5A and 5D).

In sum, these data demonstrate that transduction of AAV.STRV5-hTNNT2.PKP2a is capable of restoring PKP2 expression in iPSC PKP2-KO cardiomyocytes, resulting in a remarkable rescue of the electrical phenotype of cardiomyocytes lacking PKP2.

### Example 4: Expression of PKP2 in Wild Type Mice Upon Treatment with AAV.STRV47-hTNNT2.PKP2a

Wild type mice of 11 weeks of age were used in an in vivo pilot study in two groups (control and treated, n=4 for both groups). See Table A. The treated group was dosed with 5e13 vg/kg of AAV.STRV47-hTNNT2.PKP2a (150 μL total volume for a 1.25e12 total VG dose) by IV injection via tail vein. 21 days after administration, the expression of the human PKP2 gene in mouse cardiac tissue was assessed for both groups of mice using molecular techniques, including qPCR, RT-qPCR, and Western Blot.

TABLE A

| | | No. | | Dose | Route and | Dose Level | Study |
|---|---|---|---|---|---|---|---|
| Group | Genotype | Animals | Treatment | (vg/kg) | Volume | (total/mouse) | Length |
| 1 | WT | 4 | Vehicle | N/A | 150 μL IV | N/A | 21 Days |
| 2 | WT | 4 | AAV.STRV47- | 5e13 | 150 μL IV | 1.25e12 vg | 21 Days |
| | | | hTNNT2.PKP2a | | | | |

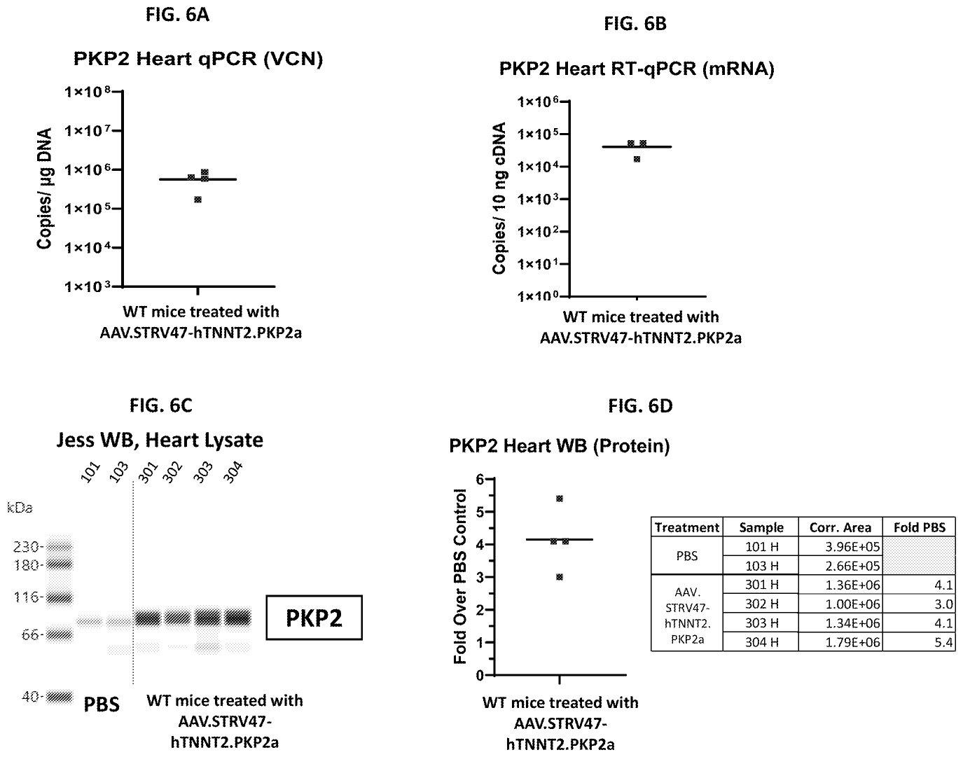

For DNA extraction, 50 mg of tissue was placed into a tube containing stainless steel beads with 300 μL of TE buffer and 30 μL of 20 mg/mL Proteinase K Solution and homogenized. Samples were then incubated at 56° C. for 20 minutes, then DNA was extracted using a Maxwell RSC Tissue DNA kit (Promega, Cat #AS1610) on a Maxwell RSC 48 instrument according to the manufacturer's protocol. DNA concentration and A260/A280 were measured using a NanoDrop. qPCR was run on a QS3 instrument (Thermo) using custom PKP2 primers and probes. FIG. 6A shows that the PKP2a transgene is detectable in the cardiac tissue of mice treated with the AAV.STRV47-hTNNT2.PKP2a vector.

For RNA extraction, 10-20 mg of tissue was placed into a tube containing stainless steel beads with 250 μL of homogenization buffer and homogenized with a bead blaster. Samples were incubated at 70° C. for 2 minutes. RNA was extracted using a Maxwell RSC Tissue RNA kit (Promega, Cat #AS1340) on a Maxwell RSC 48 instrument according to manufacturer's protocol. RNA concentration and A260/A280 were measured using a NanoDrop. For RT-qPCR, Dnase I was added to the samples and incubated at 37° C. for 2 minutes. Samples were then diluted 1:20 and the reverse transcriptase reaction was run with dNTP mix and SuperScript IV reverse transcriptase to generate cDNA. qPCR was run on a QS3 instrument (Thermo) using custom PKP2 primers and probes and normalized to a housekeeping gene, mouse GAPDH. FIG. 6B shows that PKP2a mRNA is detectable in the cardiac tissue of mice treated with the AAV.STRV47-hTNNT2.PKP2a vector.

For protein extraction, 50-100 mg of tissue sample was homogenized in PBS and protease inhibitors (Boston Bioproducts, Cat. #BP-475) using a bead blaster. Protein was detected using the Jess automated Western Blot system with anti-PKP2 antibody (1:100) from American Research Products, Inc. (Cat #03-651101), as shown in FIG. 6C. Quantification shown in FIG. 6D was conducted using the Jess software and normalized to the mean of the PBS controls. The results show that PKP2a protein is readily detectable in the cardiac tissue of mice treated with the AAV.STRV47-hTNNT2.PKP2a vector.

### Example 5: Expression of PKP2 Using AAV.STRV5-hTNNT2.PKP2a or AAV.STRV84-hTNNT2.PKP2a in Cardiac-Specific PKP2 Knockout Mouse Model (“PKP2-cKO”) Results in Prolonged Survival

A gene targeting construct was generated by introducing two loxP sites flanking mouse Pkp2 exons 2 and 3 followed by a neomycin resistance gene. The linearized construct was electroporated into mouse (C57BL/6) embryonic stem (ES) cells, followed by neomycin selection of positive ES cell clones. Positive clones were injected into mouse blastocysts, which were then injected into foster mice. The resulting F1 heterozygous mice were crossed with mice expressing flippase to excise the neomycin resistance gene. Mice homozygous for Pkp2 targeted allele (Pkp2 fl/fl) were crossed with mice expressing Cre recombinase fused to a mutant form of the human estrogen receptor ligand-binding domain under the control of a cardiomyocyte-specific promoter (αMHC-Cre-ER (T2)). Upon treatment of Pkp2 fl/fl, αMHC-Cre-ER (T2) mice with tamoxifen, Pkp2 exons 2 and 3 are deleted specifically in the heart, eliciting a substantial decrease in PKP2 protein expression. This tamoxifen-inducible, cardiomyocyte-specific deletion of mouse PKP2 protein results in a model of ARVC referred to as the PKP2-cKO mouse model.

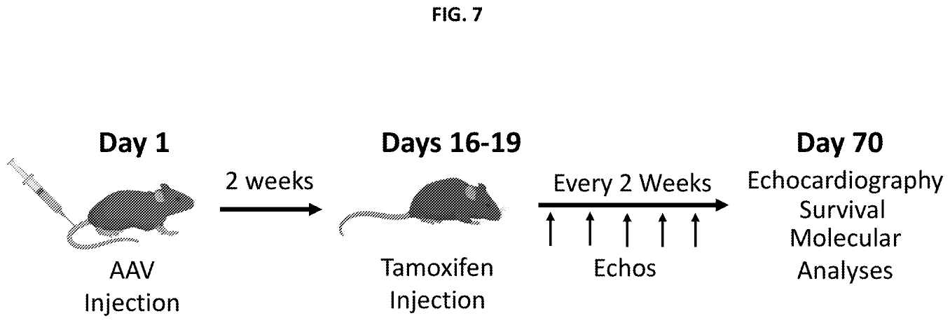

FIG. 7 shows the design for studying the treatment of a tamoxifen-induced, cardiac-specific PKP2 knockout mouse model with AAV.STRV5-hTNNT2.PKP2a or AAV.STRV84-hTNNT2.PKP2a. Mice of around 3 months of age were injected with either AAV.STRV5-hTNNT2.PKP2a or AAV.STRV84-hTNNT2.PKP2a via tail vein at 5e13vg/kg. Five groups (two positive controls: group 2 and group 3 in Table B; one negative control: group 1 in Table B; and two treated: groups 4 and 5 in Table B) were used to assess the ability to express the human PKP2 gene in mouse cardiac tissue as compared to wild type and knockout mouse PKP2 levels. See Table B.

TABLE B

| | | | | | Number of | Number of | | |
|---|---|---|---|---|---|---|---|---|
| | | Total | | | animals in | animals in | Route | |
| | | No. | | Dose | Cohort 1 | Cohort 2 | and | Test |
| Group | Genotype | Animals | Treatment | (vg/kg) | Dosed | Dosed | Volume | Duration |
| 1 | PKP2 | 10 | N/A | 0 | 5 | 5 | N/A | 70 Days |
| | fl/fl; Cre+ | | | | | | | |
| 2 | PKP2 | 10 | N/A | 0 | 5 | 5 | N/A | 70 Days |
| | wt/wt; Cre+ | | | | | | | |
| 3 | PKP2 | 10 | N/A | 0 | 5 | 5 | N/A | 70 Days |
| | fl/fl; Cre− | | | | | | | |
| 4 | PKP2 | 13 | AAV.STRV5- | 5e13 | 5 | 8 | 125 μL | 70 Days |
| | fl/fl; Cre+ | | hTNNT2.PKP2a | | | | IV | |
| 5 | PKP2 | 3 | AAV.STRV84- | 5e13 | 3 | 0 | 125 μL | 70 Days |
| | fl/fl; Cre+ | | hTNNT2.PKP2a | | | | IV | |

For cardiac-specific PKP2 deletion, mice were intraperitoneally injected 4 consecutive days with Tamoxifen (0.1 mg/g body weight) around 16 days post AAV injection. Mice were then observed for an additional 54 days to assess survival.

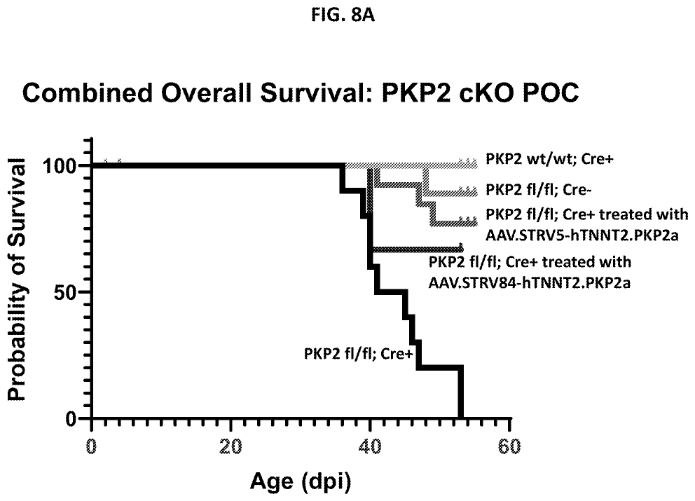

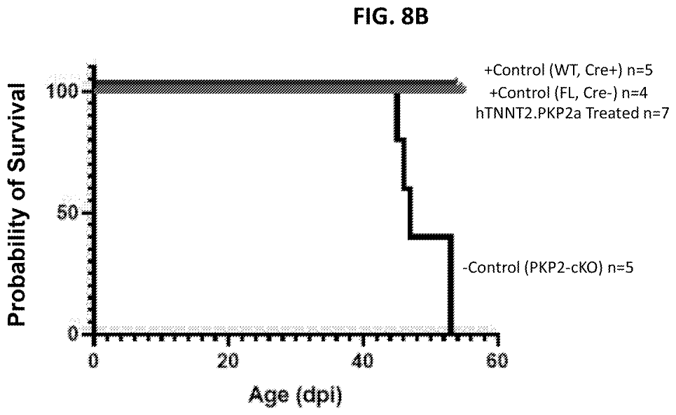

As shown in FIG. 8A and Table C, the positive control mice that are either “PKP2 wt/wt; Cre+”, or “PKP2 fl/fl; Cre-” show the highest probability of survival, with the majority of mice surviving beyond 52 days post induction (dpi). In contrast, the negative control mice that have cardiac-specific PKP2 knockout (“PKP2 fl/fl; Cre+”) survived only until about 50 days. Remarkably, the treatment of cardiac-specific PKP2 knockout mice with either AAV.STRV5-hTNNT2.PKP2a or AAV.STRV84-hTNNT2.PKP2a resulted in rescue of the PKP2 knockout phenotype, and a marked elongation in the probability of survival.

For the second cohort of Table B, cardiac-specific PKP2 knockout animals were treated with AAV.STRV5-hTNNT2.PKP2a (see Table D below) as described above and the study endpoint was day 54 or ˜8 weeks post-tamoxifen injection. The experimental design is shown in TABLE D.

In the second cohort, all unmasked animals in the positive control groups (Pkp2 fl/fl; Cre- and Pkp2 wt/wt; Cre+) survived to the scheduled necropsy endpoint, while all mice in the untreated PKP2-cKO (Pkp2 fl/fl; Cret) negative control group reached testing facility criteria for early euthanasia before the study endpoint, consistent with the disease model phenotype. Notably, all unmasked PKP2-cKO mice treated with AAV.STRV5-hTNNT2.PKP2a survived to the scheduled necropsy endpoint, indicating a treatment-mediated extension of survival. (See FIG. 8B, Tables B and D)

TABLE C

| | | | Deaths on | Deaths on |
|---|---|---|---|---|
| Group | Genotype | Treatment | Cohort 1 | Cohort 2 |
| 1 | PKP2 fl/fl; Cre+ | — | 5/5 | 5/5 |
| 2 | PKP2 wt/wt; Cre+ | — | 0/5 | 0/5 |
| 3 | PKP2 fl/fl; Cre− | — | 0/5 | 1/5 |
| 4 | PKP2 fl/fl; Cre+ | AAV.STRV5- | 2/5 | 1/8 |
| | | hTNNT2.PKP2a | | |
| 5 | PKP2 fl/fl; Cre+ | AAV.STRV84- | 1/3 | 0/0 |
| | | hTNNT2.PKP2a | | |

TABLE D

| | | | Dose per kg | Number of | Euthanasia |
|---|---|---|---|---|---|
| Treatment | ROA | Genotype | Body Weight | Animals | Timepoint* |
| None | NA | Pkp2 fl/fl; Cre+ | NA | 5 | ~8 weeks (54 days) |
| | | Pkp2 fl/fl; Cre− | NA | 5 | ~8 weeks (54 days) |
| | | Pkp2 wt/wt; Cre+ | NA | 5 | ~8 weeks (54 days) |
| AAV.STRV5- | IV | Pkp2 fl/fl; Cre+ | 5 × 10¹³ vg/kg | 8 | ~8 weeks (54 days) |
| hTNNT2.PKP2a | | | | | |

*Days post injection of tamoxifen (dpi)

### EXAMPLE 6: EXPRESSION OF PKP2 USING AAV.STRV5-HTNNT2.PKP2A OR AAV.STRV84-hTNNT2.PKP2a in Cardiac-Specific PKP2 Knockout Mouse Model

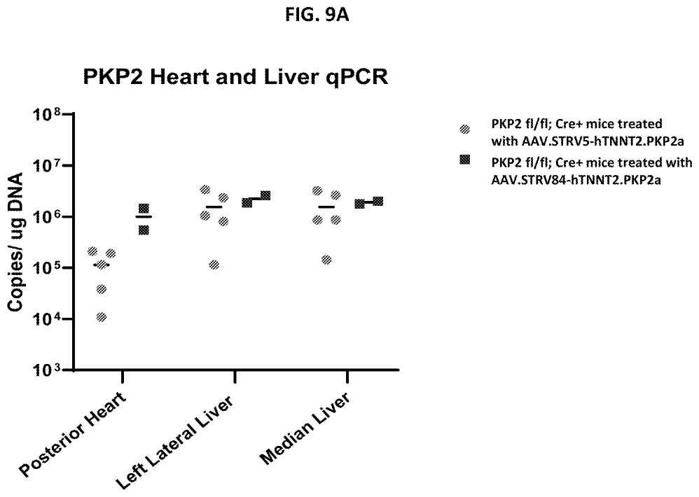

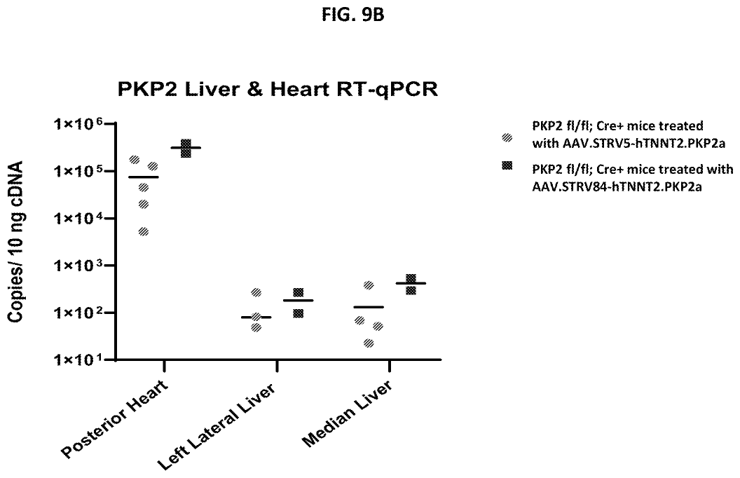

The presence of the PKP2a transgene and the expression of PKP2 mRNA in cardiac-specific PKP2 knockout mouse models upon treatment with AAV vectors comprising PKP2 gene was evaluated. DNA and RNA extraction and analyses were performed as described in Example 4 above. As shown in FIG. 9A, the results show that the PKP2a transgene is detectable in the cardiac tissue of cardiac-specific PKP2 knockout mice treated with the AAV.STRV5-hTNNT2.PKP2a vector or AAV.STRV84-hTNNT2.PKP2a vector. Also, as shown in FIG. 9B, the results show that PKP2a mRNA is detectable in the cardiac tissue of mice treated with the AAV.STRV5-hTNNT2.PKP2a vector or AAV.STRV84-hTNNT2.PKP2a vector.

Treatment with AAV.STRV84-hTNNT2.PKP2a advantageously resulted in log-fold greater copy number in the heart, as compared to treatment with AAV.STRV5-hTNNT2.PKP2a vector. Furthermore, treatment with AAV.STRV84-hTNNT2.PKP2a advantageously resulted in ½ log-fold greater mRNA expression in the heart, as compared to treatment with AAV.STRV5-hTNNT2.PKP2a vector. The copy number and mRNA expression in the liver was similar upon treatment with the two AAV vectors. Notably, FIG. 9B shows that expression from the TNNT2 promoter led to high PKP2 expression in the heart, but very low expression in the liver. This differential expression has advantageous effects in minimizing off-target effects during treatment with the AAV vectors disclosed herein.

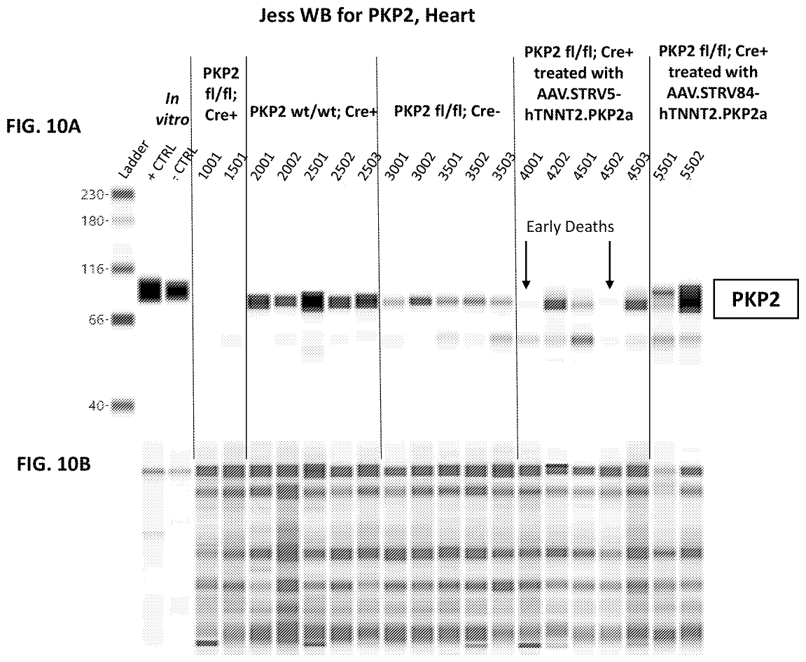

PKP2 protein expression upon treatment of PKP2 cardiac knockout mice with AAV.STRV5-hTNNT2.PKP2a vector or AAV.STRV84-hTNNT2.PKP2a vector was evaluated using methods described in Example 3 above. PKP2 Protein was detected using the Jess automated Western Blot system with anti-PKP2a-b (1:100) from ARP (Cat #03-651101), as shown in FIG. 10A, along with the total protein (loading control) in FIG. 10B. Quantification shown in Table E was conducted using the Jess software and normalized to the mean of group 2 (positive control).

As shown in FIGS. 10A and 10B and Table E, the treatment of cardiac-specific PKP2 knockout mice with either AAV.STRV5-hTNNT2.PKP2a or AAV.STRV84-hTNNT2.PKP2a leads to expression of PKP2 protein in the cardiac tissues. Furthermore, treatment with AAV.STRV84-hTNNT2.PKP2a resulted in much higher PKP2 protein expression in the heart (1.71-fold over wild type), as compared to treatment with AAV.STRV5-hTNNT2.PKP2a vector (0.52-fold over wild type), suggesting that treatment with AAV particles comprising the disclosed AAV expression cassette of SEQ ID NO: 12 and a capsid protein having the amino acid sequence of SEQ ID NO: 15 is particularly effective in rescuing PKP2 levels and function in vivo.

TABLE E

| Group | Genotype | Treatment | Fold over group 2 |
|---|---|---|---|
| 1 | PKP2 fl/fl; Cre+ | — | 0.04 |
| 2 | PKP2 wt/wt; Cre+ | — | 1 |
| 3 | PKP2 fl/fl; Cre− | — | 0.38 |
| 4 | PKP2 fl/fl; Cre+ | AAV.STRV5- | 0.52 |
| | | hTNNT2.PKP2a | |
| 5 | PKP2 fl/fl; Cre+ | AAV.STRV84- | 1.71 |
| | | hTNNT2.PKP2a | |

### Example 7: Localization of PKP2 to Desmosomes in Cardiac-Specific PKP2 Knockout Mouse Model Upon Treatment with AAV.STRV5-hTNNT2.PKP2a or AAV.STRV84-hTNNT2.PKP2a

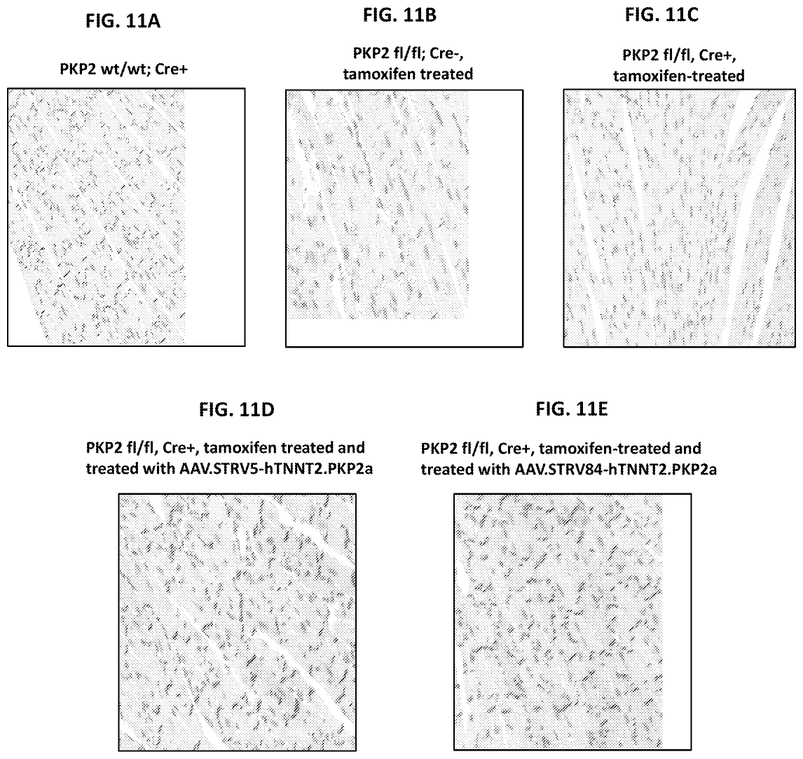

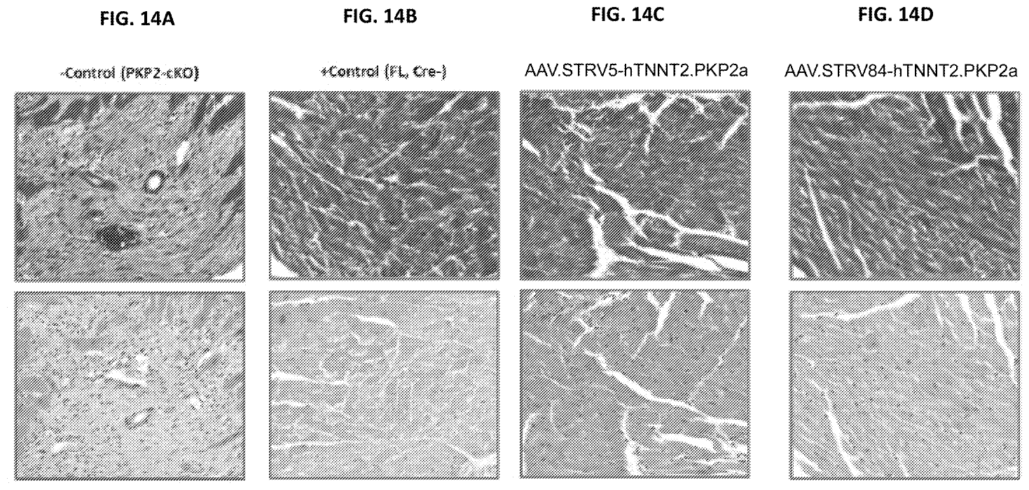

Analysis of mouse heart tissue sections by immunohistochemistry showed results consistent with the molecular analyses (FIGS. 11A-11E). Specifically, PKP2 wt/wt; Cret, and PKP2 fl/fl; Cre−, tamoxifen-treated (+Control) mice displayed PKP2 signal broadly throughout the heart (FIGS. 11A and 11B) which clearly aligned with the intercalated disc region of cardiomyocyte cell-cell junctions, consistent with PKP2 localization to the cardiac desmosome, while cardiac-specific PKP2 knockout (PKP2 fl/fl, Cre+, tamoxifen-treated; −Control) animals exhibited minimal PKP2 signal in heart tissue sections (FIG. 11C). Notably, PKP2-cKO animals treated with either AAV.STRV5-hTNNT2.PKP2a, or AAV.STRV84-hTNNT2.PKP2a after 10 weeks showed cardiac PKP2a signal throughout the heart and localization consistent with that observed in positive control animals (FIGS. 11D and 14E), providing further evidence of vector-derived PKP2a expression and confirming localization of PKP2a to the appropriate cellular region.

Taken together, the results described herein demonstrate that the treatment of mice lacking PKP2 function in cardiac tissue with AAV vectors disclosed herein, comprising PKP2a (e.g., AAV.STRV47-hTNNT2.PKP2a, AAV.STRV5-hTNNT2.PKP2a, or AAV.STRV84-hTNNT2.PKP2a) not only results in the expression of PKP2 mRNA and protein in the cardiac tissue, but also promotes accurate localization of the human PKP2 protein to desmosomes. These results also demonstrate that the expression of human PKP2 from the AAV vectors disclosed herein results in a marked improvement in the survival of animals lacking PKP2 function in cardiac tissue. Finally, the expression of human PKP2 from the AAV vectors disclosed herein is advantageously higher in heart compared to liver.

### Example 8: Cardiac Structure and Cardiac Function is Significantly Improved Upon Treatment of Cardiac-Specific PKP2 Knockout Mice with AAV.STRV5-hTNNT2.PKP2a

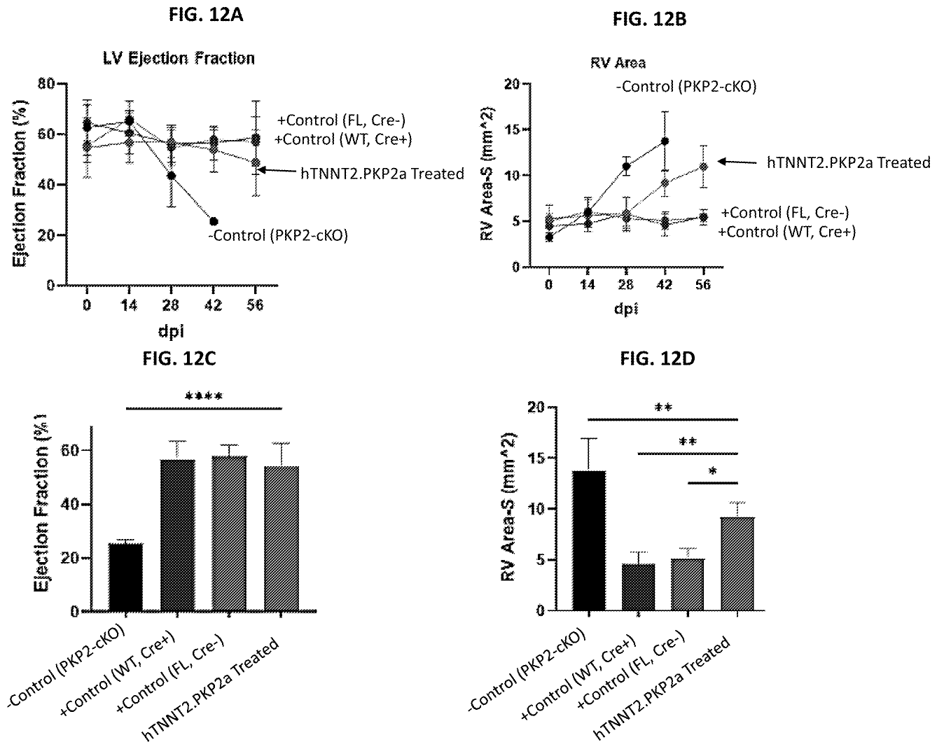

Analysis of PKP2-cKO mouse heart function and structure by echocardiography revealed a decline in left ventricular ejection fraction and an increase in right ventricular area, compared to control mice that have wild type PKP2 function (WT; Cre+ mice and floxed; Cre-mice). Remarkably, AAV.STRV5-hTNNT2.PKP2a-treated PKP2-cKO mice maintained an ejection fraction comparable to control animals and showed a delayed increase in right ventricular area (FIGS. 12A and 12B (arrows are pointing to the AAV.STRV5-hTNNT2.PKP2a treated mice)). Even at 42 days post-induction by tamoxifen, both cardiac parameters in AAV.STRV5-hTNNT2.PKP2a-treated mice were significantly improved, compared to untreated PKP2-cKO mice, indicating treatment-mediated prevention of cardiac dysfunction and delayed progression of cardiac dilatation (FIGS. 12C and 12D). In sum, these data demonstrate that AAV.STRV5-mediated expression PKP2 prevents decline of cardiac function, and delays onset and progression of cardiac dilatation.

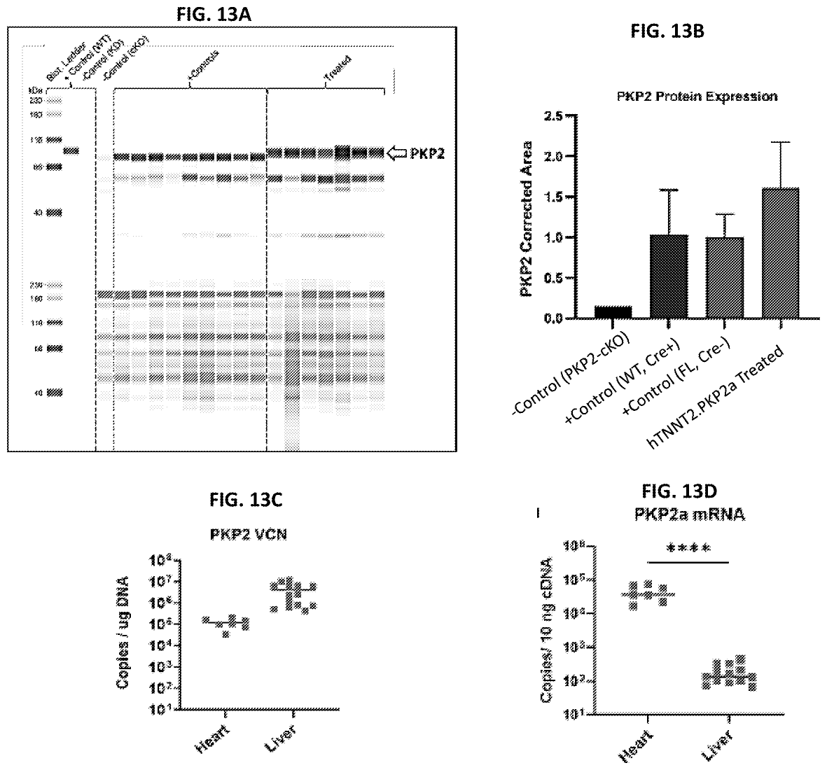

Analysis of mouse cardiac tissues by Western blot showed minimal to no expression of endogenous PKP2 in PKP2-cKO hearts, consistent with Cre-mediated deletion of Pkp2 exons 2 and 3. In contrast, PKP2-cKO mice treated with AAV.STRV5-hTNNT2.PKP2a exhibited robust human PKP2a expression in the heart at a level of approximately 1.5-fold of endogenous PKP2 levels (FIGS. 13A and 13B). Analysis of vector copy number (VCN) by qPCR showed about 10⁵ vector copies per microgram of input DNA in heart tissue and about 0.5×10⁶ vector copies per microgram of DNA in liver tissue. Further analysis of vector-derived PKP2a mRNA by RT-qPCR indicated 10⁴-10⁵ copies per 10 ng of cDNA input in heart tissue. Despite the higher VCN in liver relative to the heart, copies of PKP2a mRNA were ˜500-fold lower in liver, highlighting the cardiac-specific expression profile of the hTNNT2 promoter utilized in the cassette (FIGS. 13C and 13D). Together, these data confirm expression of vector-derived PKP2a in the mouse heart, while demonstrating minimal expression in the liver.

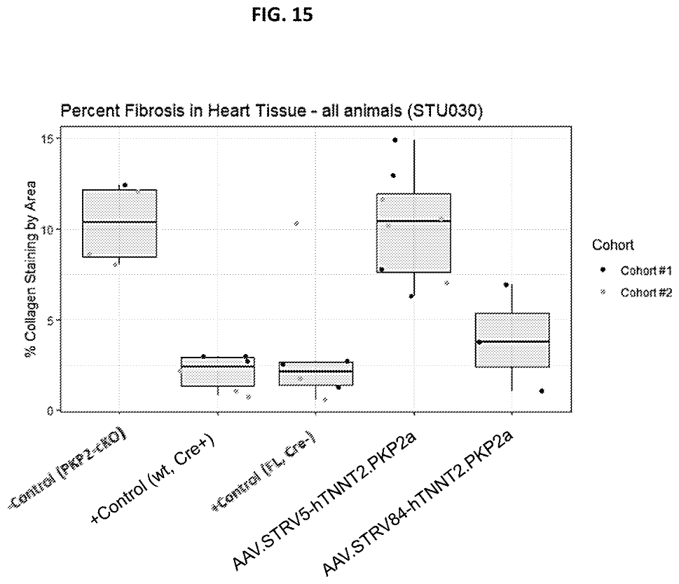

Additional analysis of cardiac tissue by Masson's trichrome staining revealed substantial collagen deposition in the right ventricles of untreated PKP2-cKO mice (FIG. 14A), indicative of pathological cardiac fibrosis. In contrast, mice with PKP2 function (+Control; FL, Cre−) exhibit minimal collagen deposition in the right ventricles, indicative of normal cardiac tissue. Remarkably, PKP2-cKO mice treated with either AAV.STRV5-hTNNT2.PKP2a or AAV.STRV84-hTNNT2.PKP2a exhibited markedly less right ventricular (RV) fibrosis (FIGS. 14C and 14D). FIG. 15 shows the quantification of fibrosis in the 2 cohorts of animals (as seen in FIGS. 8A and 8B and Table B). Treatment with AAV.STRV84-hTNNT2.PKP2a brings the fibrosis level down to almost the levels of the positive control.

In sum, the data described above clearly demonstrate that the expression of PKP2 in an AAV-mediated method (e.g. using the AAV.STRV5 or AAV.STRV84 vectors) is capable of not only rescuing the levels of PKP2 protein in a mouse model of cardiac specific PKP2-knockout, but also restoring the localization and function of PKP2 in these animals. The AAV-mediated expression of PKP2 protein described herein inhibits the formation of fibrotic regions in the cardiac tissue, thus maintaining cardiac function. Therefore, the methods disclosed herein, comprising expression of PKP2 using an AAV vector, such as AAV.STRV5 and AAV.STRV84, can alleviate the pathologies associated with loss of PKP2 function, such as in arrhythmogenic cardiomyopathy.

### Example 9: Dose-Finding and Efficacy Study in a PKP2-cKO Mouse Model

This was an 85-day dose-finding study using 79 mice. The mice were injected on day 1 with the AAV-transgene in the tail vein via an IV. On day 16, the mice were induced with tamoxifen intraperitoneally. As shown in Table F, the mice were separated into 9 groups. Group 1 are the wild-type mice. Group 2 are the PKP2 fl/fl Cre− mice. Group 3 are the PKP2 fl/fl Cre+ mice. Group 4 are the PKP2 fl/fl Cret mice that were dosed with 3e12 vg/kg of AAV.STRV84-hTNNT2.mPKP2 (mouse PKP2). Group 5 are the PKP2 fl/fl Cre+ mice that were dosed with 1e13 vg/kg of AAV.STRV84-hTNNT2.mPKP2. Group 6 are the PKP2 fl/fl Cre+ mice that were dosed with 3e13 vg/kg AAV.STRV84-hTNNT2.mPKP2. Group 7 are the PKP2 fl/fl Cre+ mice that were dosed with 1e14 vg/kg of AAV.STRV84-mTNNT2.mPKP2. Group 8 are the PKP2 fl/fl Cret mice that were dosed with 1e13 vg/kg of AAV.STRV84-hTNNT2.PKP2. Group 9 are the PKP2 fl/fl Cret mice that were dosed with 1e14 vg/kg of AAV.STRV84-hTNNT2.PKP2. In summary, in this experiment, ultra-low (UL) dose is 3e12, low (L) dose is 1e13, medium dose (M) is 3e13, and high (H) dose is 1e14 vg/kg. As reference, the selected dose for the proof-of-concept (POC) study was 5e13 vg/kg.

The mice were evaluated using biweekly echocardiography. Furthermore, heart and liver tissues from the mice were analyzed for biodistribution of the AAV vector and expression of the PKP2 transgenes, as described below.

TABLE F

| | | Total No. | | | Dose |
|---|---|---|---|---|---|
| Group | Genotype | Animals | Treatment | PKP2 | (vg/kg) |
| 1 | PKP2 wt/wt; | 8 | N/A | — | 0 |
| | Cre+ | | | | |
| 2 | PKP2 fl/fl; | 10 | N/A | — | 0 |
| | Cre− | | | | |
| 3 | PKP2 fl/fl; | 10 | N/A | — | 0 |
| | Cre+ | | | | |
| 4 | PKP2 fl/fl; | 10 | STRV84/mPKP2 | Mouse | 3e12 |
| | Cre+ | | | | |
| 5 | PKP2 fl/fl; | 10 | STRV84/mPKP2 | Mouse | 1e13 |
| | Cre+ | | | | |
| 6 | PKP2 fl/fl; | 10 | STRV84/mPKP2 | Mouse | 3e13 |
| | Cre+ | | | | |
| 7 | PKP2 fl/fl; | 10 | STRV84/mPKP2 | Mouse | 1e14 |
| | Cre+ | | | | |
| 8 | PKP2 fl/fl; | 5 | STRV84/hPKP2 | Human | 1e13 |
| | Cre+ | | | | |
| 9 | PKP2 fl/fl; | 6 | STRV84/hPKP2 | Human | 1e14 |
| | Cre+ | | | | |

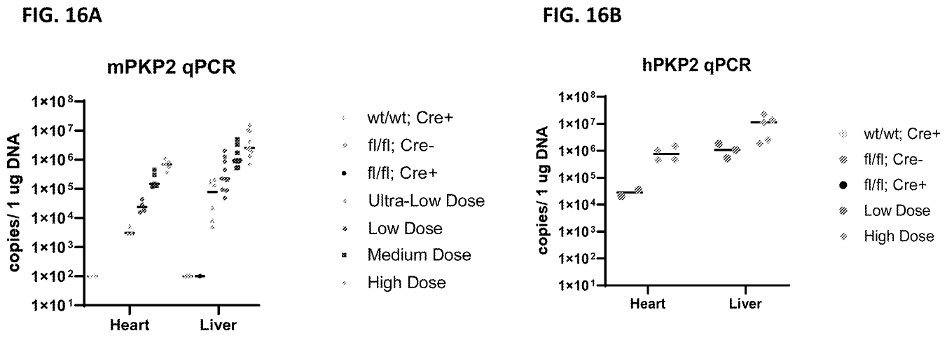

As seen in FIGS. 16A-16B, as the administered dose of human or mouse PKP2 increases, there is an increase in the vector copy number of the PKP2 DNA. This is true for both the heart and liver of the treated animals. FIG. 16A relates to data from Groups 1-7, and FIG. 16B relates to data from Groups 1-3 and 8-9.

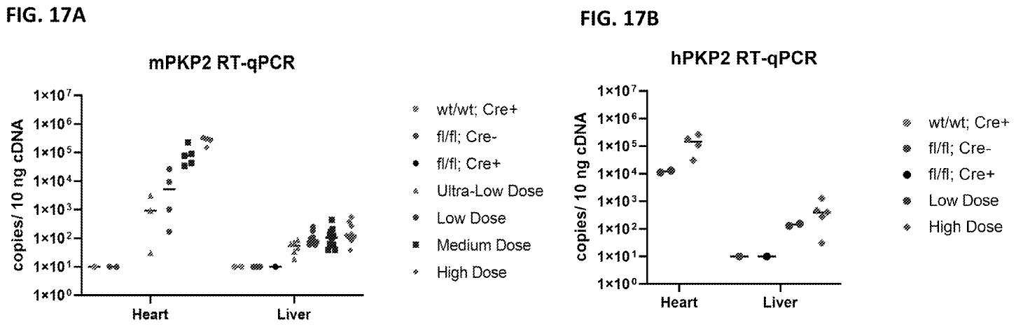

FIGS. 17A-17B show a similar correlation between dosage and PKP2 mRNA levels. However, the levels of PKP2 mRNA in the heart of the PKP2-treated animals continued to increase through the dose range, while the levels in the liver were about 1e2 copies/10 ng cDNA in all groups. Also, mPKP2 and hPKP2 had similar mRNA levels for similar dosages.

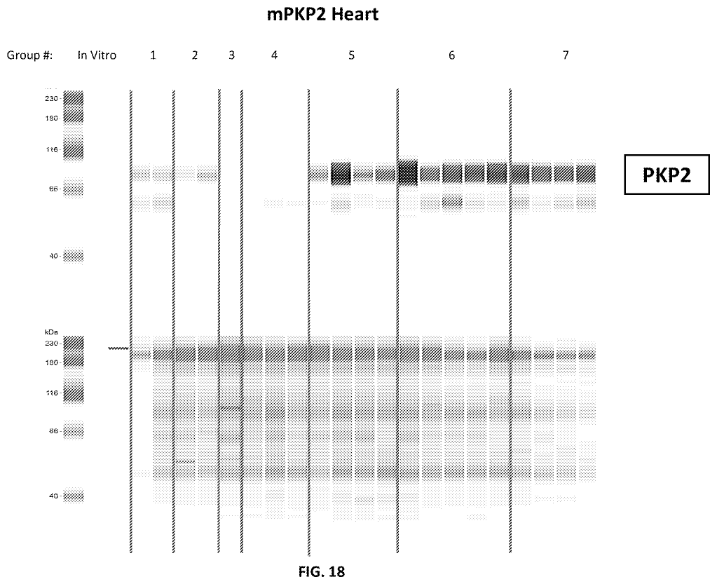

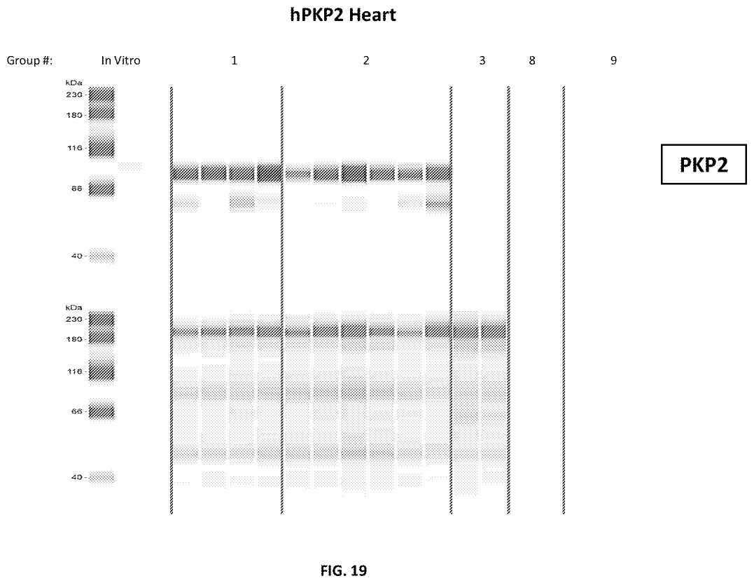

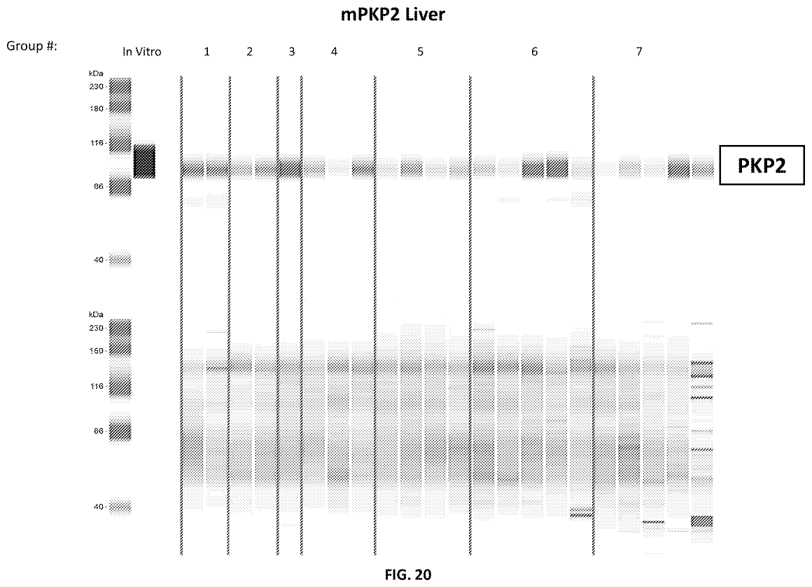

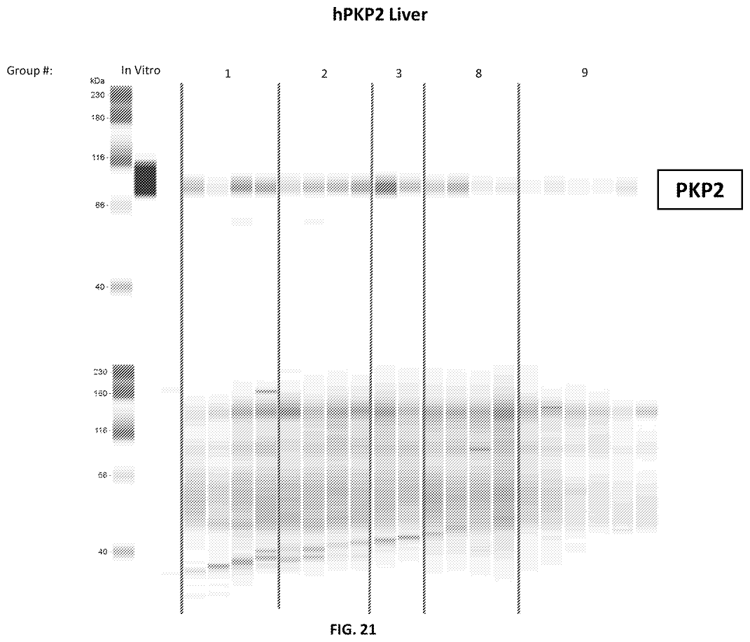

FIGS. 18-21 are western blots showing the change in PKP2 levels based on the treatment. Results for mPKP2 heart levels (FIG. 18) are shown in TABLE G. Results for hPKP2 heart levels (FIG. 19) are shown in TABLE H. Results for mPKP2 liver levels (FIG. 20) are shown in TABLE I. Results for hPKP2 liver levels (FIG. 21) are shown in TABLE J.

TABLE G

| | | | Group Average |
|---|---|---|---|
| | | Band Intensity | Band Intensity |
| | | Fold Change | Fold Change |
| Treatment | Sample | vs. Group 2 | vs. Group 2 |
| Group 2 (fl/fl; Cre−) | 2001 | n/a | 1.00 |
| | 2501 | n/a | |
| Group 1 (wt/wt; Cre+) | 1001 | 0.96 | 1.00 |
| | 1501 | 1.03 | |
| Group 3 (fl/fl; Cre+) | 3003 | 0.11 | 0.11 |
| Group 4 (ultra-low) | 4001 | 0.46 | 0.32 |
| | 4501 | 0.24 | |
| | 4505 | 0.24 | |
| Group 5 (low) | 5001 | 1.39 | 2.73 |
| | 5003 | 5.26 | (1.89 after |
| | 5501 | 1.98 | removal of |
| | 5505 | 2.29 | outliers) |
| Group 6 (medium) | 6001 | 10.73 | 5.06 |
| | 6003 | 2.40 | (3.64 after |
| | 6501 | 3.56 | removal of |
| | 6503 | 3.62 | outliers) |
| | 6505 | 4.97 | |
| Group 7 (high) | 7001 | 4.58 | 5.14 |
| | 7003 | 3.62 | (4.02 after |
| | 7501 | 3.67 | removal of |
| | 7503 | 4.21 | outliers) |
| | 7505 | 9.64 | |

TABLE H

| | | | Group Average |
|---|---|---|---|
| | | Band Intensity | Band Intensity |
| | | Fold Change | Fold Change |
| Treatment | Sample | vs. Group 2 | vs. Group 2 |
| Group 2 (fl/fl; Cre−) | 2001 | n/a | 1.00 |
| | 2003 | n/a | |
| | 2005 | n/a | |
| | 2501 | n/a | |
| | 2503 | n/a | |
| | 2505 | n/a | |
| Group 1 (wt/wt; Cre+) | 1001 | 0.81 | 0.94 |
| | 1003 | 0.86 | |
| | 1501 | 0.82 | |
| | 1503 | 1.27 | |
| Group 2 (fl/fl; Cre−) | 3003 | 0.06 | 0.08 |
| | 3502 | 0.10 | |
| Group 8 (low) | 8001 | 1.07 | 0.92 |
| | 8003 | 0.78 | |
| Group 9 (high) | 9003 | 4.20 | 4.83 |
| | 9501 | 6.15 | |
| | 9503 | 4.12 | |

TABLE I

| | | | Group Average |
|---|---|---|---|
| | | Band Intensity | Band Intensity |
| | | Fold Change | Fold Change |
| Treatment | Sample | vs. Group 2 | vs. Group 2 |
| Group 2 (fl/fl; Cre−) | 2001 | n/a | 1.00 |
| | 2501 | n/a | |
| Group 1 (wt/wt; Cre+) | 1001 | 1.08 | 1.14 |
| | 1501 | 1.20 | |
| Group 3 (fl/fl; Cre+) | 3002 | 1.67 | 1.38 |
| | 3502 | 1.09 | |
| Group 4 (ultra-low) | 4001 | 0.53 | 1.01 |
| | 4002 | 1.56 | |
| | 4501 | 0.74 | |
| | 4504 | 1.19 | |
| Group 5 (low) | 5001 | 0.76 | 0.95 |
| | 5002 | 1.23 | |
| | 5501 | 0.99 | |
| | 5502 | 0.80 | |
| Group 6 (medium) | 6001 | 2.00 | 1.26 |
| | 6002 | 2.17 | |
| | 6501 | 0.66 | |
| | 6502 | 0.21 | |
| Group 7 (high) | 7001 | 0.68 | 0.87 |
| | 7002 | 0.56 | |
| | 7501 | 1.30 | |
| | 7502 | 0.94 | |

TABLE J

| | | | Group Average |
|---|---|---|---|
| | | Band Intensity | Band Intensity |
| | | Fold Change | Fold Change |
| Treatment | Sample | vs. Group 2 | vs. Group 2 |
| Group 2 (fl/fl; Cre−) | 2001 | n/a | 1.00 |
| | 2002 | n/a | |
| | 2501 | n/a | |
| | 2502 | n/a | |
| Group 1 (wt/wt; Cre+) | 1001 | 0.79 | 0.76 |
| | 1002 | 0.47 | |
| | 1501 | 0.96 | |
| | 1502 | 0.84 | |
| Group 3 (fl/fl; Cre+) | 3002 | 1.62 | 1.3 |
| | 3502 | 0.99 | |
| Group 8 (low) | 8001 | 0.92 | 0.88 |
| | 8002 | 1.23 | |
| | 8003 | 0.71 | |
| | 8502 | 0.64 | |
| Group 9 (high) | 9001 | 0.60 | 0.52 |
| | 9002 | 0.65 | |
| | 9003 | 0.54 | |
| | 9501 | 0.44 | |
| | 9502 | 0.64 | |
| | 9503 | 0.27 | |

In conclusion, vector copy number and mRNA expression in the heart increase with increasing administered dose of AAV-PKP2. Vector copy number in the liver increases slightly with increasing dose but the mRNA levels are low and are relatively similar in all dose groups. The results show that while the while the level of PKP2 protein increases with dose in the heart, overexpression is not detected at any dose in liver. Without being bound by a theory, it is thought that these results are promoted by the liver de-targeted profile of STRV84 capsid and the use of a cardiac specific promoter. Western blot analysis of heart shows stronger expression of PKP2 with increasing doses. Expression follows this pattern across both mouse and human transgene treatment groups. Western blot analysis of liver shows similar levels of expression across all groups tested, including mPKP2 and hPKP2 treatment groups. Human vs. mouse transgene shows comparable protein expression in liver.

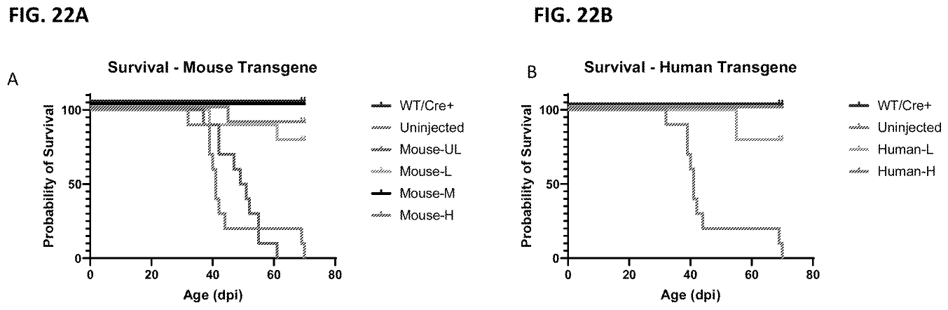

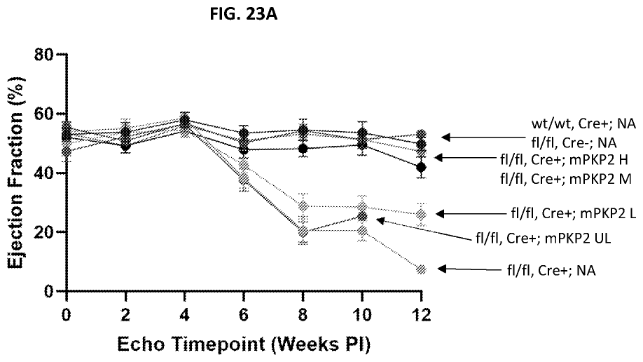

FIGS. 22A-B shows that administration of either the mouse transgene (FIG. 22A) or the human transgene (FIG. 22B) increased the probability of survival of mice lacking PKP2 function in a dose-dependent manner.

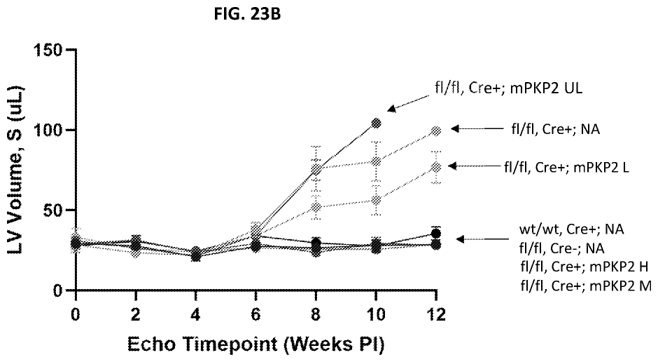

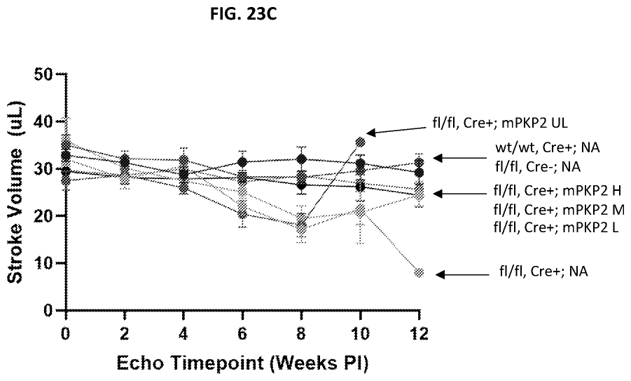

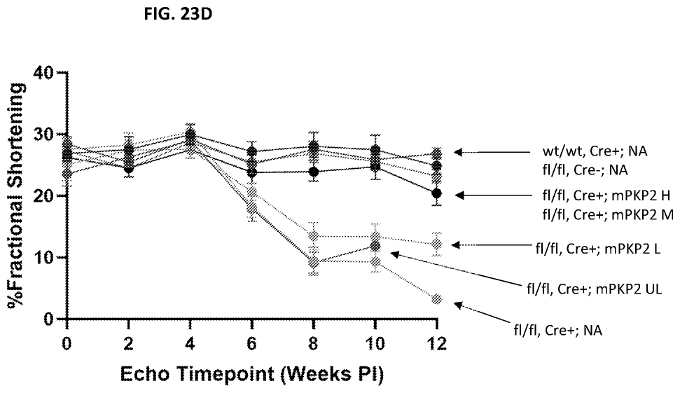

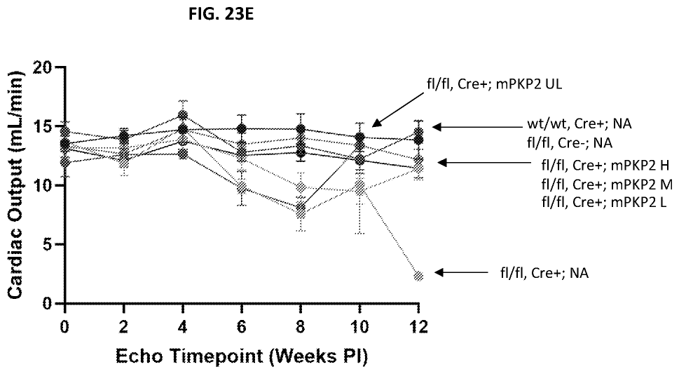

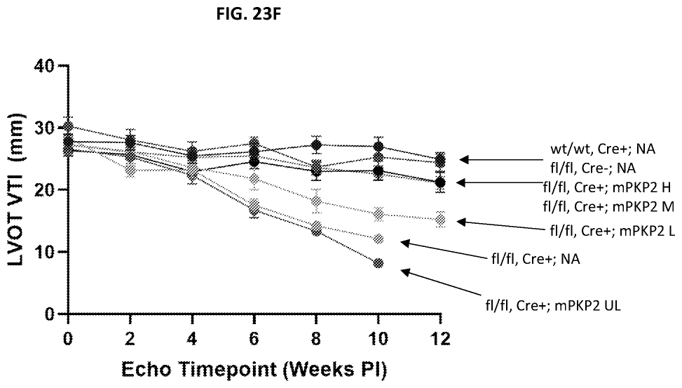

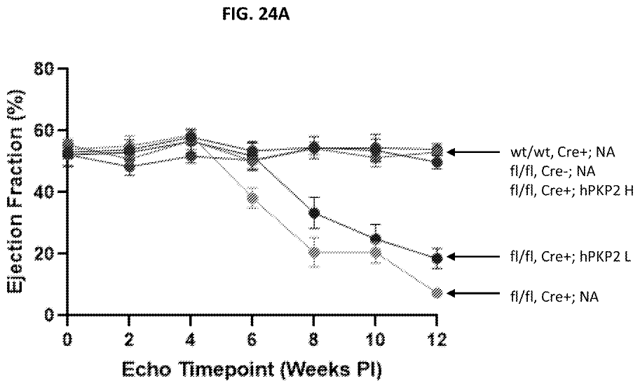

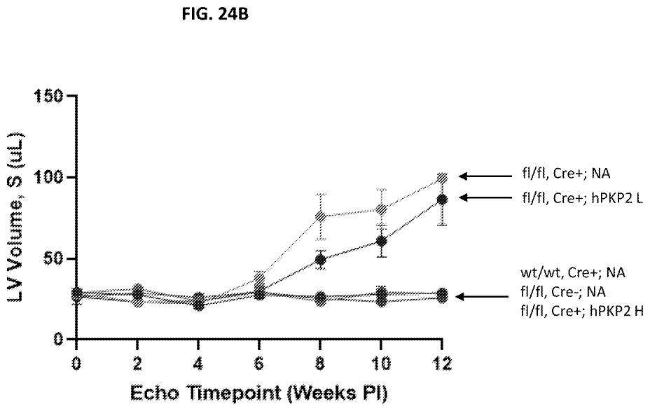

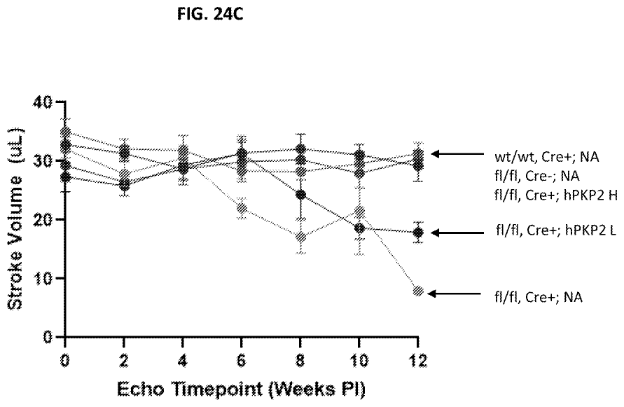

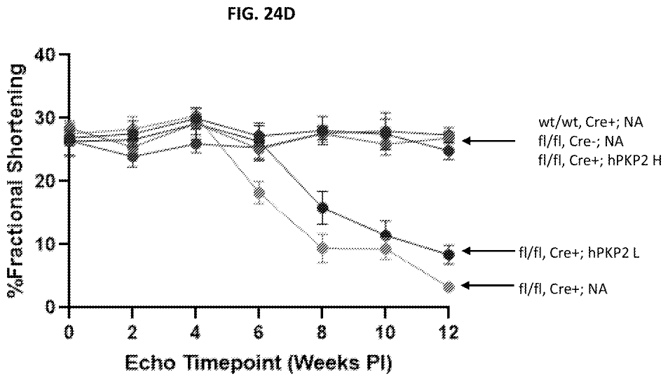

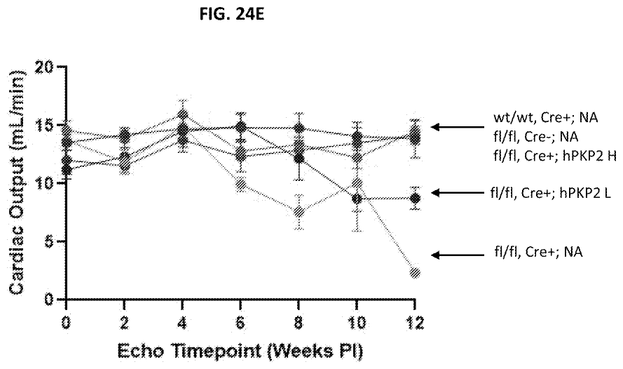

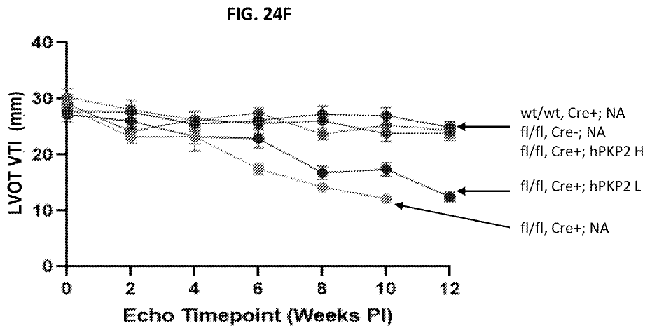

FIGS. 23A-F and 24A-F show the dose of the human transgene at which the animals exhibit a statistical improvement in health vs. untreated control mice. The echocardiography data provides a quantitative measure of cardiac structure and function. The graphs in these figures demonstrate the performance of an unaffected, healthy control heart (the two positive control groups (wt/wt, Cre+; NA and fl/fl, Cre−; NA)), as well as the impact of ARVC (the untreated, negative control group (fl/fl, Cre+; NA)), and therefore provides a gauge on how well the AAV treatment preserves cardiac structure and function. The correlation between dose and rescue of phenotype was observed for the following parameters: ejection fraction (FIG. 23A and FIG. 24A), left ventricle (LV) volume (FIG. 23B and FIG. 24B), stroke volume (FIG. 23C and FIG. 24C), percent fractional shortening (FIG. 23D and FIG. 24D), cardiac output (FIG. 23E and FIG. 24E), and left ventricular outflow tract velocity time integral (LVOT VTI) (FIG. 23F and FIG. 24F). Note all the data is from the left side of the animal.

The foregoing is illustrative of the present invention and is not to be construed as limiting thereof.

### Numbered Embodiments

The following list of embodiments is included herein for illustration purposes only and is not intended to be comprehensive or limiting. The subject matter to be claimed is expressly not limited to the following embodiments.
Embodiment 1. A nucleic acid molecule, comprising an adeno-associated virus (AAV) expression cassette, wherein the AAV expression cassette comprises, from 5′ to 3′:
   a. a 5′ AAV inverted terminal repeat (ITR);
   b. a promoter;
   c. an arrhythmogenic cardiomyopathy-associated transgene; and
   d. a 3′ AAV ITR.
Embodiment 2. The nucleic acid molecule of embodiment 1, wherein the promoter drives expression of the arrhythmogenic cardiomyopathy-associated transgene.
Embodiment 3. The nucleic acid molecule of embodiment 1 or 2, wherein the promoter is capable of expressing the transgene in a cardiac cell.
Embodiment 4. The nucleic acid molecule of any one of embodiments 1-3, wherein the promoter comprises a cardiac troponin T (TNNT2) promoter.
Embodiment 5. The nucleic acid molecule of embodiment 4, wherein the TNNT2 promoter comprises a nucleic acid sequence derived from: (i) a human TNNT2 promoter, (ii) a chicken TNNT2 promoter, (iii) a mouse TNNT2 promoter, or (iv) any combination thereof.
Embodiment 6. The nucleic acid molecule of embodiment 5, wherein the TNNT2 promoter comprises a human TNNT2 promoter.
Embodiment 7. The nucleic acid molecule of any one of embodiments 4-6, wherein the TNNT2 promoter comprises the nucleic acid sequence SEQ ID NO: 4, or a sequence at least 90% identical thereto.
Embodiment 8. The nucleic acid molecule of any one of embodiments 1-7, wherein the transgene encodes a plakophilin-2.
Embodiment 9. The nucleic acid molecule of any one of embodiments 1-8, wherein the transgene encodes a human plakophilin-2.
Embodiment 10. The nucleic acid molecule of any one of embodiments 1-9, wherein the transgene encodes an isoform 2a of human plakophilin-2.
Embodiment 11. The nucleic acid molecule of embodiment 10, wherein the isoform 2a of human plakophilin-2 comprises the amino acid sequence of SEQ ID NO: 1, or a sequence at least 90% identical thereto.
Embodiment 12. The nucleic acid molecule of any one of embodiments 1-11, wherein the transgene encodes an RNA transcript variant 2a of the PKP2 gene.
Embodiment 13. The nucleic acid molecule of any one of embodiments 1-12, wherein the transgene comprises a PKP2 gene.
Embodiment 14. The nucleic acid molecule of embodiment 13, wherein the PKP2 gene is a human PKP2 gene.
Embodiment 15. The nucleic acid molecule of any one of embodiments 1-14, wherein the transgene comprises the nucleic acid sequence of SEQ ID NO: 2, or a sequence at least 90% identical thereto.
Embodiment 16. The nucleic acid molecule of any one of embodiments 1-15, wherein at least one of the 5′ ITR and the 3′ ITR is about 110 to about 160 nucleotides in length.
Embodiment 17. The nucleic acid molecule of any one of embodiments 1-16, wherein the 5′ ITR is the same length as the 3′ ITR.
Embodiment 18. The nucleic acid molecule of any one of embodiments 1-17, wherein the 5′ ITR and the 3′ ITR have different lengths.
Embodiment 19. The nucleic acid molecule of any one of embodiments 1-18, wherein the 5′ ITR and the 3′ ITR are each about 141 nucleotides in length.
Embodiment 20. The nucleic acid molecule of any one of embodiments 1-19, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.
Embodiment 21. The nucleic acid molecule of any one of embodiments 1-20, wherein the 5′ ITR and the 3′ ITR are each isolated or derived from the genome of AAV2. Embodiment 22. The nucleic acid molecule of any one of embodiments 1-21, wherein the 5′ ITR comprises the sequence of SEQ ID NO: 5.
Embodiment 23. The nucleic acid molecule of any one of embodiments 1-22, wherein the 3′ ITR comprises the sequence of SEQ ID NO: 6.
Embodiment 24. The nucleic acid molecule of any one of embodiments 1-23, wherein the AAV expression cassette comprises an intron.
Embodiment 25. The nucleic acid molecule of embodiment 24, wherein the intron is derived from the human beta-globin gene.
Embodiment 26. The nucleic acid molecule of embodiment 24 or embodiment 25, wherein the intron comprises a nucleic acid sequence of SEQ ID NO: 7, or a sequence at least 90% identical thereto.
Embodiment 27. The nucleic acid molecule of any one of embodiments 1-26, wherein the AAV expression cassette comprises a polyadenylation signal.
Embodiment 28. The nucleic acid molecule of embodiment 27, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).
Embodiment 29. The nucleic acid molecule of embodiment 27 or embodiment 28, wherein the AAV expression cassette comprises a bGH polyadenylation signal.
Embodiment 30. The nucleic acid molecule of embodiment 29, wherein the bGH polyadenylation signal comprises a nucleic acid sequence of SEQ ID NO: 8, or a sequence at least 90% identical thereto.
Embodiment 31. The nucleic acid molecule of any one of embodiments 1-30, wherein the AAV expression cassette comprises at least one stuffer sequence.
Embodiment 32. The nucleic acid molecule of embodiment 31, wherein the at least one stuffer sequence comprises a nucleic acid sequence of SEQ ID NO: 9, or a sequence at least 90% identical thereto.
Embodiment 33. The nucleic acid molecule of embodiment 31, wherein the at least one stuffer sequence comprises a nucleic acid sequence of SEQ ID NO: 9, or a portion thereof.
Embodiment 34. The nucleic acid molecule of any one of embodiments 1-33, wherein the AAV expression cassette comprises a Kozak sequence.
Embodiment 35. The nucleic acid molecule of embodiment 34, wherein the Kozak sequence comprises a nucleic acid sequence of SEQ ID NO: 10, or a sequence at least 90% identical thereto; or a nucleic acid sequence of acagccacc, or a sequence at least 90% identical thereto.
Embodiment 36. The nucleic acid molecule of any one of embodiments 1-35, wherein the AAV expression cassette comprises an enhancer.
Embodiment 37. The nucleic acid molecule of any one of embodiments 1-36, wherein the AAV expression cassette comprises a nucleic acid sequence SEQ ID NO: 12, or a sequence at least 90% identical thereto.
Embodiment 38. A plasmid, comprising the nucleic acid molecule of any one of embodiments 1-37.
Embodiment 39. A cell, comprising the nucleic acid molecule of any one of embodiments 1-37 or the plasmid of embodiment 38.
Embodiment 40. A method of producing a recombinant AAV vector, the method comprising contacting an AAV producer cell with the nucleic acid molecule of any one of embodiments 1-37 or the plasmid of embodiment 38.
Embodiment 41. A recombinant AAV vector produced by the method of embodiment 40.
Embodiment 42. The recombinant AAV vector of embodiment 41, wherein the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.
Embodiment 43. The recombinant AAV vector of embodiment 41 or embodiment 42, wherein the recombinant AAV vector is a single-stranded AAV (ssAAV).
Embodiment 44. The recombinant AAV vector of embodiment 41 or embodiment 42, wherein the recombinant AAV vector is a self-complementary AAV (scAAV).
Embodiment 45. The recombinant AAV vector of any one of embodiments 41-44, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.
Embodiment 46. The recombinant AAV vector of any one of embodiments 41-45, wherein the AAV vector comprises a capsid protein with one or more substitutions or mutations, as compared to a wild type AAV capsid protein.
Embodiment 47. The recombinant AAV vector of any one of embodiments 41-46, wherein the AAV vector comprises a capsid protein comprising:
   a. (i) the amino acid sequence of SEQ ID NO: 13, or a sequence at least 90% identical thereto, or
   b. (ii) the amino acid sequence of SEQ ID NO: 14, or a sequence at least 90% identical thereto, or
   c. (iii) the amino acid sequence of SEQ ID NO: 15, or a sequence at least 90% identical thereto.
Embodiment 48. The recombinant AAV vector of embodiment 47, wherein the AAV vector comprises a capsid protein comprising the amino acid sequence of SEQ ID NO: 13, or a sequence at least 90% identical thereto.
Embodiment 49. The recombinant AAV vector of embodiment 48, wherein the AAV vector comprises a capsid protein comprising the amino acid sequence of SEQ ID NO: 13.
Embodiment 50. The recombinant AAV vector of embodiment 47, wherein the AAV vector comprises a capsid protein comprising the amino acid sequence of SEQ ID NO: 14, or a sequence at least 90% identical thereto.
Embodiment 51. The recombinant AAV vector of embodiment 50, wherein the AAV vector comprises a capsid protein comprising the amino acid sequence of SEQ ID NO: 14.
Embodiment 52. The recombinant AAV vector of embodiment 47, wherein the AAV vector comprises a capsid protein comprising the amino acid sequence of SEQ ID NO: 15, or a sequence at least 90% identical thereto.
Embodiment 53. The recombinant AAV vector of embodiment 52, wherein the AAV vector comprises a capsid protein comprising the amino acid sequence of SEQ ID NO: 15.
Embodiment 54. A composition, comprising: (a) the nucleic acid molecule of any one of embodiments 1-37, the plasmid of embodiment 38, the cell of embodiment 39, or the recombinant AAV vector of any one of embodiments 41-53; and (b) a pharmaceutically acceptable carrier.
Embodiment 55. A method of expressing an arrhythmogenic cardiomyopathy-associated transgene in a tissue, comprising: contacting the tissue with the nucleic acid molecule of any one of embodiments 1-37, the plasmid of embodiment 38, the recombinant AAV vector of any one of embodiments 41-53, or the composition of embodiment 54, thereby expressing the arrhythmogenic cardiomyopathy-associated transgene in the tissue.
Embodiment 56. The method of embodiment 55, wherein the tissue comprises one or more cells, and one or more desmosomal junctions.
Embodiment 57. The method of embodiment 55 or embodiment 56, wherein the one or more cells are cardiac cells, endothelial cells, skin cells, bladder cells, or gastrointestinal mucosal cells.
Embodiment 58. The method of embodiment 57, wherein the one or more cells are cardiac cells.
Embodiment 59. The method of any one of embodiments 55-58, wherein the contacting step is performed in vitro, ex vivo, or in vivo.
Embodiment 60. The method of embodiment 59, wherein the contacting step is performed in vivo in a subject in need thereof.
Embodiment 61. The method of embodiment 60, wherein the contacting step comprises administering a therapeutically effective amount of the nucleic acid molecule, the plasmid, the recombinant AAV vector, or the composition to the subject.
Embodiment 62. The method of embodiment 60 or embodiment 61, wherein the subject suffers from, or is at a risk of developing, the arrhythmogenic cardiomyopathy.
Embodiment 63. A method for treating arrhythmogenic cardiomyopathy in a subject in need thereof, comprising administering to the subject a therapeutically effective amount of the nucleic acid molecule of any one of embodiments 1-37, the plasmid of embodiment 38, the cell of embodiment 39, the recombinant AAV vector of any one of embodiments 41-53, or the composition of embodiment 54, thereby treating arrhythmogenic cardiomyopathy in the subject.
Embodiment 64. The method of embodiment 63, wherein the subject suffers from, or is at a risk of developing, the arrhythmogenic cardiomyopathy.
Embodiment 65. The method of any one of embodiments 62-64, wherein the arrhythmogenic cardiomyopathy is arrhythmogenic right ventricular cardiomyopathy.
Embodiment 66. The method of any one of embodiments 62-65, wherein the arrhythmogenic cardiomyopathy is associated with, promoted by, or caused by a genetic mutation.
Embodiment 67. The method of embodiment 66, wherein the genetic mutation comprises a mutation in the PKP2 gene.
Embodiment 68. The method of embodiment 67, wherein the mutation in the PKP2 gene results in PKP2 haploinsufficiency.
Embodiment 69. The method of any one of embodiments 62-68, wherein the method comprises diminishing the severity of; delaying the onset or progression of, and/or eliminating a symptom of the arrhythmogenic cardiomyopathy.
Embodiment 70. The method of embodiment 69, wherein the symptom of the arrhythmogenic cardiomyopathy comprises: (a) re-entrant ventricular tachycardia, (b) syncope, (c) sudden death, or (d) any combination thereof.
Embodiment 71. The method of any one of embodiments 62-70, wherein the arrhythmogenic cardiomyopathy is associated with: (a) decreased mechanical stability between cardiomyocytes of the subject, (b) disruption of gap junctions in the cardiac tissue of the subject, (c) decreased sodium currents in the cardiac tissue of the subject, (d) fibrosis of the right ventricular myocardium, or (e) any combination thereof.
Embodiment 72. The method of any one of embodiments 62-71, wherein the method comprises increasing the mechanical stability between cardiomyocytes of the subject, as compared to a control subject having arrhythmogenic cardiomyopathy, wherein the control subject has not been administered the therapeutically effective amount, or as compared to the subject prior to administration of the therapeutically effective amount.
Embodiment 73. The method of any one of embodiments 62-72, wherein the method comprises improving the function of gap junctions in the cardiac tissue of the subject, as compared to a control subject having arrhythmogenic cardiomyopathy, wherein the control subject has not been administered the therapeutically effective amount, or as compared to the subject prior to administration of the therapeutically effective amount.
Embodiment 74. The method of any one of embodiments 62-73, wherein the method comprises increasing sodium currents in the cardiac tissue of the subject, as compared to a control subject having arrhythmogenic cardiomyopathy, wherein the control subject has not been administered the therapeutically effective amount, or as compared to the subject prior to administration of the therapeutically effective amount.
Embodiment 75. The method of any one of embodiments 62-74, wherein the method comprises decreasing fibrosis of the right ventricular myocardium of the subject, as compared to a control subject having arrhythmogenic cardiomyopathy, wherein the control subject has not been administered the therapeutically effective amount, or as compared to the subject prior to administration of the therapeutically effective amount.
Embodiment 76. The method of any one of embodiments 62-75, wherein the method comprises increasing the left ventricular ejection fraction of the heart, as compared to a control subject having arrhythmogenic cardiomyopathy, wherein the control subject has not been administered the therapeutically effective amount, or as compared to the subject prior to administration of the therapeutically effective amount.
Embodiment 77. The method of any one of embodiments 62-76, wherein the method comprises decreasing the right ventricular area of the heart, as compared to a control subject having arrhythmogenic cardiomyopathy, wherein the control subject has not been administered the therapeutically effective amount, or as compared to the subject prior to administration of the therapeutically effective amount.
Embodiment 78. The method of any one of embodiments 62-77, wherein the method comprises prolonging the survival of the subject, as compared to a control subject having arrhythmogenic cardiomyopathy, wherein the control subject has not been administered the therapeutically effective amount, or as compared to the subject prior to administration of the therapeutically effective amount.
Embodiment 79. The method of any one of embodiment 60-78, wherein the subject is a human subject.
Embodiment 80. The method of any one of embodiments 60-79, wherein the nucleic acid molecule, the plasmid, the cell, the recombinant AAV vector, or composition is administered to the cardiac tissue of the subject.
Embodiment 81. The method of embodiment 80, wherein the nucleic acid molecule, the plasmid, the cell, the recombinant AAV vector, or composition is administered to the left atrium, right atrium, left ventricle, right ventricle and/or septum.
Embodiment 82. The method of any one of embodiments 60-81, wherein the nucleic acid molecule, the plasmid, the cell, the recombinant AAV vector, or composition is administered to the subject via intravenous administration, intra-arterial administration, intra-aortic administration, direct cardiac injection, coronary artery perfusion, or any combination thereof.

What is claimed is:

1. A nucleic acid molecule, comprising an adeno-associated virus (AAV) expression cassette, wherein the AAV expression cassette comprises, from 5′ to 3′:
(i) a 5′ AAV inverted terminal repeat (ITR);
(ii) a promoter;
(iii) an arrhythmogenic cardiomyopathy-associated transgene; and
(iv) a 3′ AAV ITR.

2. The nucleic acid molecule of claim 1, wherein the promoter drives expression of the arrhythmogenic cardiomyopathy-associated transgene.

3. The nucleic acid molecule of claim 1 or 2, wherein the promoter is capable of expressing the transgene in a cardiac cell.

4. The nucleic acid molecule of any one of claims 1-3, wherein the promoter comprises a cardiac troponin T (TNNT2) promoter.

5. The nucleic acid molecule of claim 4, wherein the TNNT2 promoter comprises a nucleic acid sequence derived from: (i) a human TNNT2 promoter, (ii) a chicken TNNT2 promoter, (iii) a mouse TNNT2 promoter, or (iv) any combination thereof.

6. The nucleic acid molecule of claim 5, wherein the TNNT2 promoter comprises a human TNNT2 promoter.

7. The nucleic acid molecule of any one of claims 4-6, wherein the TNNT2 promoter comprises the nucleic acid sequence SEQ ID NO: 4, or a sequence at least 90% identical thereto.

8. The nucleic acid molecule of any one of claims 1-7, wherein the transgene encodes a plakophilin-2.

9. The nucleic acid molecule of any one of claims 1-8, wherein the transgene encodes a human plakophilin-2.

10. The nucleic acid molecule of any one of claims 1-9, wherein the transgene encodes an isoform 2a of human plakophilin-2.

11. The nucleic acid molecule of claim 10, wherein the isoform 2a of human plakophilin-2 comprises the amino acid sequence of SEQ ID NO: 1, or a sequence at least 90% identical thereto.

12. The nucleic acid molecule of any one of claims 1-11, wherein the transgene encodes an RNA transcript variant 2a of the PKP2 gene.

13. The nucleic acid molecule of any one of claims 1-12, wherein the transgene comprises a PKP2 gene.

14. The nucleic acid molecule of claim 13, wherein the PKP2 gene is a human PKP2 gene.

15. The nucleic acid molecule of any one of claims 1-14, wherein the transgene comprises the nucleic acid sequence of SEQ ID NO: 2, or a sequence at least 90% identical thereto.

16. The nucleic acid molecule of any one of claims 1-15, wherein at least one of the 5′ ITR and the 3′ ITR is about 110 to about 160 nucleotides in length.

17. The nucleic acid molecule of any one of claims 1-16, wherein the 5′ ITR is the same length as the 3′ ITR.

18. The nucleic acid molecule of any one of claims 1-17, wherein the 5′ ITR and the 3′ ITR have different lengths.

19. The nucleic acid molecule of any one of claims 1-18, wherein the 5′ ITR and the 3′ ITR are each about 141 nucleotides in length.

20. The nucleic acid molecule of any one of claims 1-19, wherein at least one of the 5′ ITR and the 3′ ITR is isolated or derived from the genome of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

21. The nucleic acid molecule of any one of claims 1-20, wherein the 5′ ITR and the 3′ ITR are each isolated or derived from the genome of AAV2.

22. The nucleic acid molecule of any one of claims 1-21, wherein the 5′ ITR comprises the sequence of SEQ ID NO: 5.

23. The nucleic acid molecule of any one of claims 1-22, wherein the 3′ ITR comprises the sequence of SEQ ID NO: 6.

24. The nucleic acid molecule of any one of claims 1-23, wherein the AAV expression cassette comprises an intron.

25. The nucleic acid molecule of claim 24, wherein the intron is derived from the human beta-globin gene.

26. The nucleic acid molecule of claim 24 or claim 25, wherein the intron comprises a nucleic acid sequence of SEQ ID NO: 7, or a sequence at least 90% identical thereto.

27. The nucleic acid molecule of any one of claims 1-26, wherein the AAV expression cassette comprises a polyadenylation signal.

28. The nucleic acid molecule of claim 27, wherein the polyadenylation signal is a polyadenylation signal isolated or derived from one or more of the following genes: simian virus 40 (SV40), rBG, α-globin, β-globin, human collagen, human growth hormone (hGH), polyoma virus, human growth hormone (hGH) or bovine growth hormone (bGH).

29. The nucleic acid molecule of claim 27 or claim 28, wherein the AAV expression cassette comprises a bGH polyadenylation signal.

30. The nucleic acid molecule of claim 29, wherein the bGH polyadenylation signal comprises a nucleic acid sequence of SEQ ID NO: 8, or a sequence at least 90% identical thereto.

31. The nucleic acid molecule of any one of claims 1-30, wherein the AAV expression cassette comprises at least one stuffer sequence.

32. The nucleic acid molecule of claim 31, wherein the at least one stuffer sequence comprises a nucleic acid sequence of SEQ ID NO: 9, or a sequence at least 90% identical thereto.

33. The nucleic acid molecule of claim 31, wherein the at least one stuffer sequence comprises a nucleic acid sequence of SEQ ID NO: 9, or a portion thereof.

34. The nucleic acid molecule of any one of claims 1-33, wherein the AAV expression cassette comprises a Kozak sequence.

35. The nucleic acid molecule of claim 34, wherein the Kozak sequence comprises a nucleic acid sequence of SEQ ID NO: 10, or a sequence at least 90% identical thereto; or a nucleic acid sequence of acagccacc, or a sequence at least 90% identical thereto.

36. The nucleic acid molecule of any one of claims 1-35, wherein the AAV expression cassette comprises an enhancer.

37. The nucleic acid molecule of any one of claims 1-36, wherein the AAV expression cassette comprises a nucleic acid sequence SEQ ID NO: 12, or a sequence at least 90% identical thereto.

38. A plasmid, comprising the nucleic acid molecule of any one of claims 1-37.

39. A cell, comprising the nucleic acid molecule of any one of claims 1-37 or the plasmid of claim 38.

40. A method of producing a recombinant AAV vector, the method comprising contacting an AAV producer cell with the nucleic acid molecule of any one of claims 1-37 or the plasmid of claim 38.

41. A recombinant AAV vector produced by the method of claim 40.

42. The recombinant AAV vector of claim 41, wherein the vector is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.

43. The recombinant AAV vector of claim 41 or claim 42, wherein the recombinant AAV vector is a single-stranded AAV (ssAAV).

44. The recombinant AAV vector of claim 41 or claim 42, wherein the recombinant AAV vector is a self-complementary AAV (scAAV).

45. The recombinant AAV vector of any one of claims 41-44, wherein the AAV vector comprises a capsid protein of AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV or Bovine AAV.

46. The recombinant AAV vector of any one of claims 41-45, wherein the AAV vector comprises a capsid protein with one or more substitutions or mutations, as compared to a wild type AAV capsid protein.

47. The recombinant AAV vector of any one of claims 41-46, wherein the AAV vector comprises a capsid protein comprising:
a. (i) the amino acid sequence of SEQ ID NO: 13, or a sequence at least 90% identical thereto, or
b. (ii) the amino acid sequence of SEQ ID NO: 14, or a sequence at least 90% identical thereto, or
c. (iii) the amino acid sequence of SEQ ID NO: 15, or a sequence at least 90% identical thereto.

48. The recombinant AAV vector of claim 47, wherein the AAV vector comprises a capsid protein comprising the amino acid sequence of SEQ ID NO: 13, or a sequence at least 90% identical thereto.

49. The recombinant AAV vector of claim 48, wherein the AAV vector comprises a capsid protein comprising the amino acid sequence of SEQ ID NO: 13.

50. The recombinant AAV vector of claim 47, wherein the AAV vector comprises a capsid protein comprising the amino acid sequence of SEQ ID NO: 14, or a sequence at least 90% identical thereto.

51. The recombinant AAV vector of claim 50, wherein the AAV vector comprises a capsid protein comprising the amino acid sequence of SEQ ID NO: 14.

52. The recombinant AAV vector of claim 47, wherein the AAV vector comprises a capsid protein comprising the amino acid sequence of SEQ ID NO: 15, or a sequence at least 90% identical thereto.

53. The recombinant AAV vector of claim 52, wherein the AAV vector comprises a capsid protein comprising the amino acid sequence of SEQ ID NO: 15.

54. A composition, comprising: (a) the nucleic acid molecule of any one of claims 1-37, the plasmid of claim 38, the cell of claim 39, or the recombinant AAV vector of any one of claims 41-53; and (b) a pharmaceutically acceptable carrier.

55. A method of expressing an arrhythmogenic cardiomyopathy-associated transgene in a tissue, comprising: contacting the tissue with the nucleic acid molecule of any one of claims 1-37, the plasmid of claim 38, the recombinant AAV vector of any one of claims 41-53, or the composition of claim 54, thereby expressing the arrhythmogenic cardiomyopathy-associated transgene in the tissue.

56. The method of claim 55, wherein the tissue comprises one or more cells, and one or more desmosomal junctions.

57. The method of claim 55 or claim 56, wherein the one or more cells are cardiac cells, endothelial cells, skin cells, bladder cells, or gastrointestinal mucosal cells.

58. The method of claim 57, wherein the one or more cells are cardiac cells.

59. The method of any one of claims 55-58, wherein the contacting step is performed in vitro, ex vivo, or in vivo.

60. The method of claim 59, wherein the contacting step is performed in vivo in a subject in need thereof.

61. The method of claim 60, wherein the contacting step comprises administering a therapeutically effective amount of the nucleic acid molecule, the plasmid, the recombinant AAV vector, or the composition to the subject.

62. The method of claim 60 or claim 61, wherein the subject suffers from, or is at a risk of developing, the arrhythmogenic cardiomyopathy.

63. A method for treating arrhythmogenic cardiomyopathy in a subject in need thereof, comprising administering to the subject a therapeutically effective amount of the nucleic acid molecule of any one of claims 1-37, the plasmid of claim 38, the cell of claim 39, the recombinant AAV vector of any one of claims 41-53, or the composition of claim 54, thereby treating arrhythmogenic cardiomyopathy in the subject.

64. The method of claim 63, wherein the subject suffers from, or is at a risk of developing, the arrhythmogenic cardiomyopathy.

65. The method of any one of claims 62-64, wherein the arrhythmogenic cardiomyopathy is arrhythmogenic right ventricular cardiomyopathy.

66. The method of any one of claims 62-65, wherein the arrhythmogenic cardiomyopathy is associated with, promoted by, or caused by a genetic mutation.

67. The method of claim 66, wherein the genetic mutation comprises a mutation in the PKP2 gene.

68. The method of claim 67, wherein the mutation in the PKP2 gene results in PKP2 haploinsufficiency.

69. The method of any one of claims 62-68, wherein the method comprises diminishing the severity of; delaying the onset or progression of; and/or eliminating a symptom of the arrhythmogenic cardiomyopathy.

70. The method of claim 69, wherein the symptom of the arrhythmogenic cardiomyopathy comprises: (a) re-entrant ventricular tachycardia, (b) syncope, (c) sudden death, or (d) any combination thereof.

71. The method of any one of claims 62-70, wherein the arrhythmogenic cardiomyopathy is associated with: (a) decreased mechanical stability between cardiomyocytes of the subject, (b) disruption of gap junctions in the cardiac tissue of the subject, (c) decreased sodium currents in the cardiac tissue of the subject, (d) fibrosis of the right ventricular myocardium, or (e) any combination thereof.

72. The method of any one of claims 62-71, wherein the method comprises increasing the mechanical stability between cardiomyocytes of the subject, as compared to a control subject having arrhythmogenic cardiomyopathy, wherein the control subject has not been administered the therapeutically effective amount, or as compared to the subject prior to administration of the therapeutically effective amount.

73. The method of any one of claims 62-72, wherein the method comprises improving the function of gap junctions in the cardiac tissue of the subject, as compared to a control subject having arrhythmogenic cardiomyopathy, wherein the control subject has not been administered the therapeutically effective amount, or as compared to the subject prior to administration of the therapeutically effective amount.

74. The method of any one of claims 62-73, wherein the method comprises increasing sodium currents in the cardiac tissue of the subject, as compared to a control subject having arrhythmogenic cardiomyopathy, wherein the control subject has not been administered the therapeutically effective amount, or as compared to the subject prior to administration of the therapeutically effective amount.

75. The method of any one of claims 62-74, wherein the method comprises decreasing fibrosis of the right ventricular myocardium of the subject, as compared to a control subject having arrhythmogenic cardiomyopathy, wherein the control subject has not been administered the therapeutically effective amount, or as compared to the subject prior to administration of the therapeutically effective amount.

76. The method of any one of claims 62-75, wherein the method comprises increasing the left ventricular ejection fraction of the heart, as compared to a control subject having arrhythmogenic cardiomyopathy, wherein the control subject has not been administered the therapeutically effective amount, or as compared to the subject prior to administration of the therapeutically effective amount.

77. The method of any one of claims 62-76, wherein the method comprises decreasing the right ventricular area of the heart, as compared to a control subject having arrhythmogenic cardiomyopathy, wherein the control subject has not been administered the therapeutically effective amount, or as compared to the subject prior to administration of the therapeutically effective amount.

78. The method of any one of claims 62-77, wherein the method comprises prolonging the survival of the subject, as compared to a control subject having arrhythmogenic cardiomyopathy, wherein the control subject has not been administered the therapeutically effective amount, or as compared to the expected survival of the subject prior to administration of the therapeutically effective amount.

79. The method of any one of claim 60-78, wherein the subject is a human subject.

80. The method of any one of claims 60-79, wherein the nucleic acid molecule, the plasmid, the cell, the recombinant AAV vector, or composition is administered to the cardiac tissue of the subject.

81. The method of claim 80, wherein the nucleic acid molecule, the plasmid, the cell, the recombinant AAV vector, or composition is administered to the left atrium, right atrium, left ventricle, right ventricle and/or septum.

82. The method of any one of claims 60-81, wherein the nucleic acid molecule, the plasmid, the cell, the recombinant AAV vector, or composition is administered to the subject via intravenous administration, intra-arterial administration, intra-aortic administration, direct cardiac injection, coronary artery perfusion, or any combination thereof.


SEQUENCE LISTING (20 sequences)

SEQ ID NO: 1 - protein, 837 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 2 - DNA, 2514 bp, synthetic construct
  misc_feature 1..2514: note = PKP2a Transgene sequence
  source 1..2514: mol_type = other DNA
atggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactga

SEQ ID NO: 3 - protein, 881 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITDHTVNLRSRNGWPGAVAHACNPSTLGGQGGRITRSGVRDQPDQHGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 4 - DNA, 544 bp, synthetic construct
  misc_feature 1..544: note = pTNNT2 - cardiac troponin T (TNNT2) promoter
  source 1..544: mol_type = other DNA
ctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcag

SEQ ID NO: 5 - DNA, 141 bp, synthetic construct
  misc_feature 1..141: note = 5' AAV ITR
  source 1..141: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 6 - DNA, 141 bp, synthetic construct
  misc_feature 1..141: note = 3' AAV ITR
  source 1..141: mol_type = other DNA
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 7 - DNA, 476 bp, synthetic construct
  misc_feature 1..476: note = hBGin - human beta-globin gene intron
  source 1..476: mol_type = other DNA
gtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacag

SEQ ID NO: 8 - DNA, 225 bp, synthetic construct
  misc_feature 1..225: note = bovine growth hormone (bGH) polyadenylation signal
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 9 - DNA, 2165 bp, synthetic construct
  misc_feature 1..2165: note = hAlbumin stuffer sequence
  source 1..2165: mol_type = other DNA
gcgcgccatcgatgtgaagtgggtaacctttatttcccttctttttctctttagctcggcttattccaggggtgtgtttcgtcgagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacctatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgtcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacctttttgaaaaaatacttatacgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagacttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgc

SEQ ID NO: 10 - DNA, 12 bp, synthetic construct
  misc_feature 1..12: note = Kozak sequence
  source 1..12: mol_type = other DNA
acccggagcagc

SEQ ID NO: 11 - skipped

SEQ ID NO: 12 - DNA, 4186 bp, synthetic construct
  misc_feature 1..4186: note = AAV expression cassette
  source 1..4186: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacactagtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggttggttgtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggcgccgtgaattcaccgcgggcccgatccaccggtcgccgccaccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgagtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaatcgatgttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 13 - protein, 736 aa, synthetic construct
  REGION 1..736: note = AAV mutant capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTIGVSLGGGQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 14 - protein, 736 aa, synthetic construct
  REGION 1..736: note = AAV mutant capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTRQDQPINAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 15 - protein, 736 aa, synthetic construct
  REGION 1..736: note = AAV mutant capsid protein
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQNAVGALSTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - DNA, 301 bp, synthetic construct
  misc_feature 1..301: note = enhancer sequence
  source 1..301: mol_type = other DNA
tacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 17 - DNA, 122 bp, synthetic construct
  misc_feature 1..122: note = poly A signal
  source 1..122: mol_type = other DNA
taagatacattgatgagtttggacaaaccacaactagaatgcagtgaaaaaaatgctttatttgtgaaatttgtgatgctattgctttatttgtaaccattataagctgcaataaacaagtt

SEQ ID NO: 18 - DNA, 56 bp, synthetic construct
  misc_feature 1..56: note = poly A signal
  source 1..56: mol_type = other DNA
aataaaggaaatttattttcattgcaatagtgtgttggaattttttgtgtctctca

SEQ ID NO: 19 - DNA, 4060 bp, synthetic construct
  source 1..4060: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgcgtcgacactagtctcagtccattaggagccagtagcctggaagatgtctttacccccagcatcagttcaagtggagcagcacataactcttgccctctgccttccaagattctggtgctgagacttatggagtgtcttggaggttgccttctgccccccaaccctgctcccagctggccctcccaggcctgggttgctggcctctgctttatcaggattctcaagagggacagctggtttatgttgcatgactgttccctgcatatctgctctggttttaaatagcttatctgagcagctggaggaccacatgggcttatatggcgtggggtacatgttcctgtagccttgtccctggcacctgccaaaatagcagccaacaccccccacccccaccgccatccccctgccccacccgtcccctgtcgcacattcctccctccgcagggctggctcaccaggccccagcccacatgcctgcttaaagccctctccatcctctgcctcacccagtccccgctgagactgagcagacgcctccaggatctgtcggcaggttggttgtgagtctatgggacccttgatgttttctttccccttcttttctatggttaagttcatgtcataggaaggggagaagtaacagggtacacatattgaccaaatcagggtaattttgcatttgtaattttaaaaaatgctttcttcttttaatatacttttttgtttatcttatttctaatactttccctaatctctttctttcagggcaataatgatacaatgtatcatgcctctttgcaccattctaaagaataacagtgataatttctgggttaaggcaatagcaatatttctgcatataaatatttctgcatataaattgtaactgatgtaagaggtttcatattgctaatagcagctacaatccagctaccattctgcttttattttatggttgggataaggctggattattctgagtccaagctaggcccttttgctaatcatgttcatacctcttatcttcctcccacagggcgccgtgaattcaccgcgggcccgatccaccggtcgccgccaccatggccgtccccggctcactggccgagtgtggctacatccggactgtgctgggccagcagatcctgggtcacctggacagctccagcctggccttgccctccgaggccagactgaggctggccggcagcagcggccgcggcgacccggcggcccggagccagcggatccaggagcaggtgcagcagaccctggcccgccggggccggagctctgcggtcagcgggaaccttcaccgaaccagcagtgtccctgagtatgtctacaagctacacgtggttgagaatgactttgttggacggcagtcacctgtcactagggactatgacatgcttaaggctggaatgactgccacttatggaagtcgctgggggagagcagcagcacagtacagttcccagaagtcagtggaggagagatcctggaggcagcctctgaggagacttgagatttccccagatagcagcccggagagagcccactatgggcacagcgaataccagtatgcctggcggagccacgtggtgcctggtgggcgcctcaccctgccacgctatgctcgctcagagatcctgggcctacgccaggctggcacagcccgcaggccacctgggtgcgggtcattcagcgatgctgtcttcgacaatggcccactcaagcccacaatgcccacccaccctcctggcaccagccacagtgcgggcagcttgttggaagagaccactgtgcgcgtgagccaggctcggcttcaaagcacgcagagcagaaccgcccgctcctcctggcccaggagctcagtccgcagtagcctgcgggagccaggaagaatgctgaccaccgcaggccaggctgccgtgggcagcggggatgcacatggggacaggagtgtcttcgctgacgcccagctggggaatgcagacatcgaaatgaccttggagagagctgtgaatatgctggatgcagaccatgtaccagtatccaagatctctgccgcagctaccttcatacagcatgagagcttccaaaaatctgaagcacggaaaagggtcaatcaacttcgaggcatccctaagcttctacaactgctcaaacttcagaacgaagatgttcaacgggctgcttgtggggccttgagaaacttggtatttgaagacaatgataacaagttggaggtagctgaactgaatggagtgcctcggctactgcaggtgctgaagcaaaccagagacttggagacaaaaaagcaaataacaggtttgctctggaacctgtcctctagtgataaactcaagcatctcatgataacagaagccttgctcaccttgacagagagtgtcatcatccccttctccgggtggcctgaaggcgactaccccaaagccaatggcttgcttgattttgatatattctacaatgtcactggatgcctaaggaacatgagctctgctggccccgatgggaggaagatgatgagaaggtgtgatggtctcattgactcattggtccactatgtcagaggaactattgcagactaccagccagatgataaggccacagagaactgtgtgtgcattcttcataacctctcctaccagctggaggcagagctcccagagaaatattcccagagtatctacatgcaaaaccggaatatccagactaacagtaacaaaagtattgggtgttttggcagccgaagtaggaaactaaaagagcaataccaagacttgcagatgccagaggaaaggagcaatccacatggcattgagtggctgtggcattccattgtgataaggatgtatttgtccttaattgccaagagtacccgaaactatacccaagaggcatcacttggggctctccagaacctcacagcaggaggcggcccgatacccacattggtggctcgaatggttgtccaaaaggaaaatggtcttcagcatacacggaagatgctgcacgtgggtgatcccagtgtgaaaaagactgcggtctccctgctgaggaatttgtcacggaatctttccctgcagaatgaaattgctaaagaaactctaccagatttggtttctataattcctgacacagtcccaagtactgaccttctcattgaaaccacagcctctgcttgctatacgttgaacaatttaatgcaaaatagttaccagaatgcacgagaccttctgaacaccggaggtctgcagaaaattatgaccatcagcataggggaaggctatgcccccaacaaggccagcaaagcagcctctgtcctcctttactctctgtgggcacatacagagctgcatcacgcctataagaaggctcagtttaagaaaacagactttgtcaacagccggactgccaaagcctaccactctcttaaagactgagtttaaacaagcttggtaccgtgattaatcttcgaatgactgacctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaatcgatgttggttttgtaagctttaccagatgaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 20 - DNA, 2388 bp, synthetic construct
  source 1..2388: mol_type = other DNA
atggccgtccccggctcactggccgagtgtggctacatccggactgtgctgggccagcagatcctgggtcacctggacagctccagcctggccttgccctccgaggccagactgaggctggccggcagcagcggccgcggcgacccggcggcccggagccagcggatccaggagcaggtgcagcagaccctggcccgccggggccggagctctgcggtcagcgggaaccttcaccgaaccagcagtgtccctgagtatgtctacaagctacacgtggttgagaatgactttgttggacggcagtcacctgtcactagggactatgacatgcttaaggctggaatgactgccacttatggaagtcgctgggggagagcagcagcacagtacagttcccagaagtcagtggaggagagatcctggaggcagcctctgaggagacttgagatttccccagatagcagcccggagagagcccactatgggcacagcgaataccagtatgcctggcggagccacgtggtgcctggtgggcgcctcaccctgccacgctatgctcgctcagagatcctgggcctacgccaggctggcacagcccgcaggccacctgggtgcgggtcattcagcgatgctgtcttcgacaatggcccactcaagcccacaatgcccacccaccctcctggcaccagccacagtgcgggcagcttgttggaagagaccactgtgcgcgtgagccaggctcggcttcaaagcacgcagagcagaaccgcccgctcctcctggcccaggagctcagtccgcagtagcctgcgggagccaggaagaatgctgaccaccgcaggccaggctgccgtgggcagcggggatgcacatggggacaggagtgtcttcgctgacgcccagctggggaatgcagacatcgaaatgaccttggagagagctgtgaatatgctggatgcagaccatgtaccagtatccaagatctctgccgcagctaccttcatacagcatgagagcttccaaaaatctgaagcacggaaaagggtcaatcaacttcgaggcatccctaagcttctacaactgctcaaacttcagaacgaagatgttcaacgggctgcttgtggggccttgagaaacttggtatttgaagacaatgataacaagttggaggtagctgaactgaatggagtgcctcggctactgcaggtgctgaagcaaaccagagacttggagacaaaaaagcaaataacaggtttgctctggaacctgtcctctagtgataaactcaagcatctcatgataacagaagccttgctcaccttgacagagagtgtcatcatccccttctccgggtggcctgaaggcgactaccccaaagccaatggcttgcttgattttgatatattctacaatgtcactggatgcctaaggaacatgagctctgctggccccgatgggaggaagatgatgagaaggtgtgatggtctcattgactcattggtccactatgtcagaggaactattgcagactaccagccagatgataaggccacagagaactgtgtgtgcattcttcataacctctcctaccagctggaggcagagctcccagagaaatattcccagagtatctacatgcaaaaccggaatatccagactaacagtaacaaaagtattgggtgttttggcagccgaagtaggaaactaaaagagcaataccaagacttgcagatgccagaggaaaggagcaatccacatggcattgagtggctgtggcattccattgtgataaggatgtatttgtccttaattgccaagagtacccgaaactatacccaagaggcatcacttggggctctccagaacctcacagcaggaggcggcccgatacccacattggtggctcgaatggttgtccaaaaggaaaatggtcttcagcatacacggaagatgctgcacgtgggtgatcccagtgtgaaaaagactgcggtctccctgctgaggaatttgtcacggaatctttccctgcagaatgaaattgctaaagaaactctaccagatttggtttctataattcctgacacagtcccaagtactgaccttctcattgaaaccacagcctctgcttgctatacgttgaacaatttaatgcaaaatagttaccagaatgcacgagaccttctgaacaccggaggtctgcagaaaattatgaccatcagcataggggaaggctatgcccccaacaaggccagcaaagcagcctctgtcctcctttactctctgtgggcacatacagagctgcatcacgcctataagaaggctcagtttaagaaaacagactttgtcaacagccggactgccaaagcctaccactctcttaaagactga